(19) United States
(12) Patent Application Publication
(10) Pub. No.: US 2014/0288185 A1
(43) Pub. Date: Sep. 25, 2014

(54) NICOTINIC RECEPTOR NON-COMPETITIVE MODULATORS

(71) Applicant: Targacept, Inc., Winston-Salem, NC (US)

(72) Inventors: Srinivasa Rao Akireddy, Winston-Salem, NC (US); Jason Speake, Winston-Salem, NC (US); Balwinder Singh Bhatti, Winston-Salem, NC (US); Daniel Yohannes, Winston-Salem, NC (US); John Genus, Winston-Salem, NC (US); Yunde Xiao, Clemmons, NC (US)

(21) Appl. No.: 14/355,316

(22) PCT Filed: Nov. 1, 2012
(86) PCT No.: PCT/US2012/062940

§ 371 Date: Apr. 30, 2014

Related U.S. Application Data
(60) Provisional application No. 61/554,998, filed on Nov. 3, 2011.

(51) Int. Cl. C07C 211/38; C07C 215/42

(52) U.S. Cl. CPC C07C 211/38; C07C 215/42
USPC 514/662; 564/459

(57) ABSTRACT

The present invention relates to compounds that modulate nicotinic receptors as non-competitive antagonists, methods for their synthesis, methods for use, and their pharmaceutical compositions.

### FIELD OF THE INVENTION

The present invention relates to compounds that modulate nicotinic receptors as non-competitive modulators (e.g., non-competitive antagonists), methods for their synthesis, methods for use, and their pharmaceutical compositions.

### BACKGROUND OF THE INVENTION

Nicotinic receptors are targets for a great number of exogenous and endogenous compounds that allosterically modulate their function. See, Arias, H. R., Binding sites for exogenous and endogenous non-competitive inhibitors of the nicotinic acetylcholine receptor, Biochimica et Biophysica Acta—Reviews on Biomembranes 1376: 173-220 (1998) and Arias, H. R., Bhumireddy, P., Anesthetics as chemical tools to study the structure and function of nicotinic acetylcholine receptors, Current Protein & Peptide Science 6: 451-472 (2005). The function of nicotinic receptors can be decreased or blocked by structurally different compounds called non-competitive modulators, including non-competitive antagonists (reviewed by Arias, H. R., Bhumireddy, P., Bouzat, C., Molecular mechanisms and binding site locations for noncompetitive antagonists of nicotinic acetylcholine receptors. The International Journal of Biochemistry & Cell Biology 38: 1254-1276 (2006)).

Non-competitive modulators comprise a wide range of structurally different compounds that inhibit receptor function by acting at a site or sites different from the orthosteric binding site. Receptor modulation has proved to be highly complex. The mechanisms of action and binding affinities of non-competitive modulators differ among nicotinic receptor subtypes (Arias et al., 2006). Non-competitive modulators may act by at least two different mechanisms: an allosteric and/or a steric mechanism.

An allosteric antagonist mechanism involves the binding of a non-competitive antagonist to the receptor and stabilization of a non-conducting conformational state, namely, a resting or desensitized state, and/or an increase in the receptor desensitization rate.

In contrast, a straightforward representation of a steric mechanism is that an antagonist molecule physically blocks the ion channel. Such antagonists may be termed non-competitive channel modulators (NCMs). Some inhibit the receptors by binding within the pore when the receptor is in the open state, thereby physically blocking ion permeation. While some act only as pure open-channel blockers, others block both open and closed channels. Such antagonists inhibit ion flux through a mechanism that does not involve binding at the orthosteric sites.

Barbiturates, dissociative anesthetics, antidepressants, and certain steroids have been shown to inhibit nicotinic receptors by allosteric mechanisms, including open and closed channel blockade. Studies of barbiturates support a model whereby binding occurs to both open and closed states of the receptors, resulting in blockade of the flow of ions. See, Dilger, J. P., Boguslaysky, R., Barann, M., Katz, T., Vidal, A. M., Mechanisms of barbiturate inhibition of acetylcholine receptor channels, Journal General Physiology 109: 401-414 (1997). Although the inhibitory action of local anesthetics on nerve conduction is primarily mediated by blocking voltage-gated sodium channels, nicotinic receptors are also targets of local anesthetics. See, Arias, H. R., Role of local anesthetics on both cholinergic and serotonergic ionotropic receptors, Neuroscience and Biobehavioral Reviews 23: 817-843 (1999) and Arias, H. R. & Blanton, M. P., Molecular and physicochemical aspects of local anesthetics acting on nicotinic acetylcholine receptor-containing membranes, Mini Reviews in Medicinal Chemistry 2: 385-410 (2002).

For example, tetracaine binds to the receptor channels preferentially in the resting state. Dissociative anesthetics inhibit several neuronal-type nicotinic receptors in clinical concentration ranges, with examples such as phencyclidine (PCP) (Connolly, J., Boulter, J., & Heinemann, S. F., Alpha 4-beta 2 and other nicotinic acetylcholine receptor subtypes as targets of psychoactive and addictive drugs, British Journal of Pharmacology 105: 657-666 (1992)), ketamine (Flood, P. & Krasowski M. D., Intravenous anesthetics differentially modulate ligand-gated ion channels, Anesthesiology 92: 1418-1425 (2000); and Ho, K. K. & Flood, P., Single amino acid residue in the extracellular portion of transmembrane segment 2 in the nicotinic α7 acetylcholine receptor modulates sensitivity to ketamine, Anesthesiology 100: 657-662 (2004)), and dizocilpine (Krasowski, M. D., & Harrison, N. L., General anaesthetic actions on ligand-gated ion channels, Cellular and Molecular Life Sciences 55: 1278-1303 (1999)). Studies indicate that the dissociative anesthetics bind to a single or overlapping sites in the resting ion channel, and suggest that the ketamine/PCP locus partially overlaps the tetracaine binding site in the receptor channel. Dizocilpine, also known as MK-801, is a dissociative anesthetic and anticonvulsant which also acts as a non-competitive antagonist at different nicotinic receptors. Dizocilpine is reported to be an open-channel blocker of α4β2 neuronal nicotinic receptors. See, Buisson, B., & Bertrand, D., Open-channel blockers at the human α4β2 neuronal nicotinic acetylcholine receptor, Molecular Pharmacology 53: 555-563 (1998).

In addition to their well-known actions on monoamine and serotonin reuptake systems, antidepressants have also been shown to modulate nicotinic receptors. Early studies showed that tricyclic antidepressants act as non-competitive antagonists. See, Gumilar, F., Arias, H. R., Spitzmaul, G., Bouzat, C., Molecular mechanisms of inhibition of nicotinic acetylcholine receptors by tricyclic antidepressants. Neuropharmacology 45: 964-76 (2003). Garćia-Colunga et al., report that fluoxetine, a selective serotonin reuptake inhibitor (SSRI), inhibits membrane currents elicited by activation of muscle or neuronal nicotinic receptors in a non-competitive manner; either by increasing the rate of desensitization and/or by inducing channel blockade. See, Garćia-Colunga, J., Awad, J. N., & Miledi, R., Blockage of muscle and neuronal nicotinic acetylcholine receptors by fluoxetine (Prozac), Proceedings of the National Academy of Sciences USA 94: 2041-2044 (1997); and Garćia-Colunga, J., Vazquez-Gomez, E., & Miledi, R., Combined actions of zinc and fluoxetine on nicotinic acetylcholine receptors, The Pharmacogenomics Journal 4: 388-393 (2004). Mecamylamine, previously approved for the treatment of hypertension, is a classical non-competitive nicotinic receptor antagonist, and is also well known to inhibit receptor function by blocking the ion channel. See, Giniatullin, R. A., Sokolova, E. M., Di Angelantonio, S., Skorinkin, A., Talantova, M. V., Nistri, A. Rapid Relief of Block by Mecamylamine of Neuronal Nicotinic Acetylcholine Receptors of Rat Chromaffin Cells In Vitro: An Electrophysiological and Modeling Study. Molecular Pharmacology 58: 778-787 (2000).

### SUMMARY OF THE INVENTION

The present invention includes compounds of Formula I:

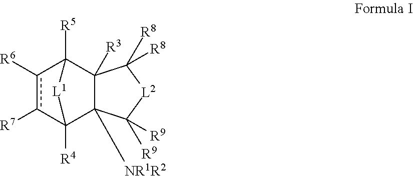

wherein

each of R¹ and R² individually is H, C₁₋₆ alkyl, or aryl-substituted C₁₋₆ alkyl, or R¹ and R² combine with the nitrogen atom to which they are attached to form a 3- to 8-membered ring, which ring may be optionally substituted with C₁₋₆ alkyl, aryl, C₁₋₆ alkoxy, or aryloxy substituents;

R³ is H, C₁₋₆ alkyl, hydroxyl-substituted C₁₋₆ alkyl, or C₁₋₆ alkoxy-substituted C₁₋₆ alkyl;

each of R⁴, R⁵, R⁶, and R⁷ individually is H, C₁₋₆ alkyl, or C₁₋₆ alkoxy;

each R⁸ individually is H, C₁₋₆ alkyl, or C₁₋₆ alkoxy;

each R⁹ individually is H, C₁₋₆ alkyl, or C₁₋₆ alkoxy;

each L¹ and L² individually is a linker species selected from the group consisting of CR¹⁰R¹¹, CR¹⁰R¹¹CR¹²R¹³, and O;

each of R¹⁰, R¹¹, R¹², and R¹³ individually is hydrogen or C₁₋₆ alkyl;

or a pharmaceutically acceptable salt thereof.

The present invention includes pharmaceutical compositions comprising a compound of the present invention or a pharmaceutically acceptable salt thereof. The pharmaceutical compositions of the present invention can be used for treating or preventing a wide variety of conditions or disorders, and particularly those disorders characterized by dysfunction of nicotinic cholinergic neurotransmission or the degeneration of the nicotinic cholinergic neurons.

The present invention includes a method for treating or preventing disorders and dysfunctions, such as CNS disorders and dysfunctions, and also for treating or preventing certain conditions, for example, alleviating pain, hypertension, and inflammation, in mammals in need of such treatment. The methods involve administering to a subject a therapeutically effective amount of a compound of the present invention, including a salt thereof, or a pharmaceutical composition that includes such compounds.

### DETAILED DESCRIPTION OF THE INVENTION

### I. Compounds

One embodiment of the present invention includes compounds of Formula I:

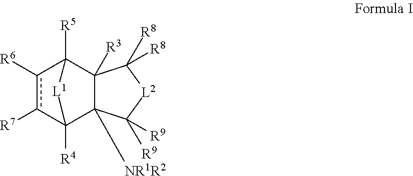

wherein

each of R¹ and R² individually is H, C₁₋₆ alkyl, or aryl-substituted C₁₋₆ alkyl, or R¹ and R² combine with the nitrogen atom to which they are attached to form a 3- to 8-membered ring, which ring may be optionally substituted with C₁₋₆ alkyl, aryl, C₁₋₆ alkoxy, or aryloxy substituents;

R³ is H, C₁₋₆ alkyl, hydroxyl-substituted C₁₋₆ alkyl, or C₁₋₆ alkoxy-substituted C₁₋₆ alkyl;

each of R⁴, R⁵, R⁶, and R⁷ individually is H, C₁₋₆ alkyl, or C₁₋₆ alkoxy;

each R⁸ individually is H, C₁₋₆ alkyl, or C₁₋₆ alkoxy;

each R⁹ individually is H, C₁₋₆ alkyl, or C₁₋₆ alkoxy;

each L¹ and L² individually is a linker species selected from the group consisting of CR¹⁰R¹¹, CR¹⁰R¹¹CR¹²R¹³, and O;

each of R¹⁰, R¹¹, R¹², and R¹³ individually is hydrogen or C₁₋₆ alkyl;

or a pharmaceutically acceptable salt thereof.

In one embodiment, a compound is selected from the group consisting of N,7a-dimethyloctahydro-4,7-methano-1H-inden-3a-amine and stereoisomers thereof, or a pharmaceutical acceptable salt thereof.

In one embodiment, the compound is (3aS,4S,7R,7aS)-N,7a-dimethyloctahydro-4,7-methano-1H-inden-3a-amine or a pharmaceutically acceptable salt thereof.

In one embodiment, the compound is (3aR,4R,7S,7aR)-N,7a-dimethyloctahydro-4,7-methano-1H-inden-3a-amine or a pharmaceutically acceptable salt thereof.

One aspect of the present invention includes a pharmaceutical composition comprising a compound of the present invention and a pharmaceutically acceptable carrier.

One aspect of the present invention includes a method for the treatment or prevention of a disease or condition mediated by a neuronal nicotinic receptor, specifically through the use of non-competitive modulators (e.g., non-competitive antagonists), including but not limited channel blockers, comprising the administration of a compound of the present invention. In one embodiment, the disease or condition is a CNS disorder. In another embodiment, the disease or condition is inflammation or an inflammatory response. In another embodiment, the disease or condition is pain. In another embodiment, the disease or condition is neovascularization. In another embodiment, the disease or condition is hypertension. In another embodiment, the disease or condition is another disorder described herein.

One aspect of the present invention includes use of a compound of the present invention for the preparation of a medicament for the treatment or prevention of a disease or condition mediated by a neuronal nicotinic receptor, specifically through the use of non-competitive antagonists, such as channel blockers. In one embodiment, the disease or condition is a CNS disorder. In another embodiment, the disease or condition is inflammation or an inflammatory response. In another embodiment, the disease or condition is pain. In another embodiment, the disease or condition is neovascularization. In another embodiment, the disease or condition is hypertension. In another embodiment, the disease or condition is another disorder described herein.

One aspect of the present invention includes a compound of the present invention for use as an active therapeutic substance. One aspect, thus, includes a compound of the present invention for use in the treatment or prevention of a disease or condition mediated by a neuronal nicotinic receptor, specifically through the use of non-competitive antagonists, such as channel blockers. In one embodiment, the disease or condition is a CNS disorder. In another embodiment, the disease or condition is inflammation or an inflammatory response. In another embodiment, the disease or condition is pain. In another embodiment, the disease or condition is neovascularization. In another embodiment, the disease or condition is hypertension. In another embodiment, the disease or condition is another disorder described herein.

Particular diseases or conditions include depression, including major depressive disorder, hypertension, irritable bowel syndrome (IBS), including IBS-D (diarrhea predominant), over active bladder (OAB), and addiction, including smoking cessation.

The scope of the present invention includes all combinations of aspects and embodiments.

The following definitions are meant to clarify, but not limit, the terms defined. If a particular term used herein is not specifically defined, such term should not be considered indefinite. Rather, terms are used within their accepted meanings.

As used throughout this specification, the preferred number of atoms, such as carbon atoms, will be represented by, for example, the phrase “C_{x-y} alkyl,” which refers to an alkyl group, as herein defined, containing the specified number of carbon atoms. Similar terminology will apply for other preferred terms and ranges as well. Thus, for example, C₁₋₆ alkyl represents a straight or branched chain hydrocarbon containing one to six carbon atoms.

As used herein the term “alkyl” refers to a straight or branched chain hydrocarbon, which may be optionally substituted, with multiple degrees of substitution being allowed. Examples of “alkyl” as used herein include, but are not limited to, methyl, ethyl, propyl, isopropyl, isobutyl, n-butyl, tert-butyl, isopentyl, and n-pentyl.

As used herein, the terms “methylene,” “ethylene,” and “ethenylene,” refer to divalent forms —CH₂—, —CH₂—CH₂—, and —CH═CH—.

As used herein, the term “aryl” refers to a single benzene ring or fused benzene ring system which may be optionally substituted, with multiple degrees of substitution being allowed. Examples of “aryl” groups as used include, but are not limited to, phenyl, 2-naphthyl, 1-naphthyl, anthracene, and phenanthrene. Preferable aryl rings have five- to ten-members.

As used herein, a fused benzene ring system encompassed within the term “aryl” includes fused polycyclic hydrocarbons, namely where a cyclic hydrocarbon with less than maximum number of noncumulative double bonds, for example where a saturated hydrocarbon ring (cycloalkyl, such as a cyclopentyl ring) is fused with an aromatic ring (aryl, such as a benzene ring) to form, for example, groups such as indanyl and acenaphthalenyl, and also includes such groups as, for non-limiting examples, dihydronaphthalene and tetrahydronaphthalene.

As used herein the term “alkoxy” refers to a group —OR^{a}, where R^{a} is alkyl as herein defined.

As used herein the term “aryloxy” refers to a group —OR^{a}, where R^{a} is aryl as herein defined.

As used herein “amino” refers to a group —NR^{a}R^{b}, where each of R^{a} and R^{b} is hydrogen. Additionally, “substituted amino” refers to a group —NR^{a}R^{b} wherein each of R^{a} and R^{b} individually is alkyl, arylalkyl or aryl. As used herein, when either R^{a} or R^{b} is other than hydrogen, such a group may be referred to as a “substituted amino” or, for example if R^{a} is H and R^{b} is alkyl, as an “alkylamino.”

As used herein, the term “pharmaceutically acceptable” refers to carrier(s), diluent(s), excipient(s) or salt forms of the compounds of the present invention that are compatible with the other ingredients of the formulation and not deleterious to the recipient of the pharmaceutical composition.

As used herein, the term “pharmaceutical composition” refers to a compound of the present invention optionally admixed with one or more pharmaceutically acceptable carriers, diluents, or excipients. Pharmaceutical compositions preferably exhibit a degree of stability to environmental conditions so as to make them suitable for manufacturing and commercialization purposes.

As used herein, the terms “effective amount”, “therapeutic amount”, and “effective dose” refer to an amount of the compound of the present invention sufficient to elicit the desired pharmacological or therapeutic effects, thus resulting in an effective treatment of a disorder. Treatment of a disorder may be manifested by delaying or preventing the onset or progression of the disorder, as well as the onset or progression of symptoms associated with the disorder. Treatment of a disorder may also be manifested by a decrease or elimination of symptoms, reversal of the progression of the disorder, as well as any other contribution to the well being of the patient.

The effective dose can vary, depending upon factors such as the condition of the patient, the severity of the symptoms of the disorder, and the manner in which the pharmaceutical composition is administered. Typically, to be administered in an effective dose, compounds may be administered in an amount of less than 5 mg/kg of patient weight. The compounds may be administered in an amount from less than about 1 mg/kg patient weight to less than about 100 μg/kg of patient weight, and further between about 1 μg/kg to less than 100 μg/kg of patient weight. The foregoing effective doses typically represent that amount that may be administered as a single dose, or as one or more doses that may be administered over a 24 hours period.

The compounds of this invention may be made by a variety of methods, including well-established synthetic methods. Illustrative general synthetic methods are set out below and then specific compounds of the invention are prepared in the working Examples.

In the examples described below, protecting groups for sensitive or reactive groups are employed where necessary in accordance with general principles of synthetic chemistry. Protecting groups are manipulated according to standard methods of organic synthesis (T. W. Green and P. G. M. Wuts (1999) Protecting Groups in Organic Synthesis, 3^{rd} Edition, John Wiley & Sons, herein incorporated by reference with regard to protecting groups). These groups are removed at a convenient stage of the compound synthesis using methods that are readily apparent to those skilled in the art. The selection of processes as well as the reaction conditions and order of their execution shall be consistent with the preparation of compounds of the present invention.

The present invention also provides a method for the synthesis of compounds useful as intermediates in the preparation of compounds of the present invention along with methods for their preparation.

The compounds can be prepared according to the methods described below using readily available starting materials and reagents. In these reactions, variants may be employed which are themselves known to those of ordinary skill in this art but are not described in detail here.

Unless otherwise stated, structures depicted herein are also meant to include compounds which differ only in the presence of one or more isotopically enriched atoms. Compounds having the present structure except for the replacement of a hydrogen atom by a deuterium or tritium, or the replacement of a carbon atom by a ¹³C- or ¹⁴C-enriched carbon are within the scope of the invention. For example, deuterium has been widely used to examine the pharmacokinetics and metabolism of biologically active compounds. Although deuterium behaves similarly to hydrogen from a chemical perspective, there are significant differences in bond energies and bond lengths between a deuterium-carbon bond and a hydrogen-carbon bond. Consequently, replacement of hydrogen by deuterium in a biologically active compound may result in a compound that generally retains its biochemical potency and selectivity but manifests significantly different absorption, distribution, metabolism, and/or excretion (ADME) properties compared to its isotope-free counterpart. Thus, deuterium substitution may result in improved drug efficacy, safety, and/or tolerability for some biologically active compounds.

The compounds of the present invention may crystallize in more than one form, a characteristic known as polymorphism, and such polymorphic forms (“polymorphs”) are within the scope of the present invention. Polymorphism generally can occur as a response to changes in temperature, pressure, or both. Polymorphism can also result from variations in the crystallization process. Polymorphs can be distinguished by various physical characteristics known in the art such as x-ray diffraction patterns, solubility, and melting point.

Certain of the compounds described herein contain one or more chiral centers, or may otherwise be capable of existing as multiple stereoisomers. The scope of the present invention includes mixtures of stereoisomers as well as purified enantiomers or enantiomerically/diastereomerically enriched mixtures. Also included within the scope of the invention are the individual isomers of the compounds represented by the formulae of the present invention, as well as any wholly or partially equilibrated mixtures thereof. The present invention also includes the individual isomers of the compounds represented by the formulas above as mixtures with isomers thereof in which one or more chiral centers are inverted.

When a compound is desired as a single enantiomer, such may be obtained by stereospecific synthesis, by resolution of the final product or any convenient intermediate, or by chiral chromatographic methods as are known in the art. Resolution of the final product, an intermediate, or a starting material may be effected by any suitable method known in the art. See, for example, Stereochemistry of Organic Compounds (Wiley-Interscience, 1994).

The stereochemical designations are assigned herein in accordance with the order of elution of the compounds as disclosed in PCT/US2011/037634, herein incorporated by reference.

The present invention includes a salt or solvate of the compounds herein described, including combinations thereof such as a solvate of a salt. The compounds of the present invention may exist in solvated, for example hydrated, as well as unsolvated forms, and the present invention encompasses all such forms.

Typically, but not absolutely, the salts of the present invention are pharmaceutically acceptable salts. Salts encompassed within the term “pharmaceutically acceptable salts” refer to non-toxic salts of the compounds of this invention.

Examples of suitable pharmaceutically acceptable salts include inorganic acid addition salts such as chloride, bromide, sulfate, phosphate, and nitrate; organic acid addition salts such as acetate, galactarate, propionate, succinate, lactate, glycolate, malate, tartrate, citrate, maleate, fumarate, methanesulfonate, p-toluenesulfonate, and ascorbate; salts with acidic amino acid such as aspartate and glutamate; alkali metal salts such as sodium salt and potassium salt; alkaline earth metal salts such as magnesium salt and calcium salt; ammonium salt; organic basic salts such as trimethylamine salt, triethylamine salt, pyridine salt, picoline salt, dicyclohexylamine salt, and N,N′-dibenzylethylenediamine salt; and salts with basic amino acid such as lysine salt and arginine salt. The salts may be in some cases hydrates or ethanol solvates.

Those of skill in the art of organic chemistry will appreciate that more than one systematic name can be given to many organic compounds. Thus, Compound VII, representative of the present invention and shown in Scheme 1, can be named N,7a-dimethyloctahydro-4,7-methano-1H-inden-3a-amine. Compound VII can also be named 3,7a-dimethylhexahydro-4,7-methanoindan-3a-amine or N,6-dimethyltricyclo[5.2.1.0^{2,6}]decan-2-amine. The scope of the present invention should not be considered as lacking clarity due to the several potential naming conventions possible for the compounds.

### II. General Synthetic Methods

Those skilled in the art of organic synthesis will appreciate that there exist multiple means of producing compounds of the present invention, as well as means for producing compounds of the present invention which are labeled with a radioisotope appropriate to various uses.

One means of producing compounds of the present invention is outlined in Scheme 1. Thus, norcamphor (2-norbornanone) can be alkylated adjacent to the carbonyl functionality, using techniques well known to those of skill in the art of organic synthesis. Typically, treatment of the ketone with strong base (e.g., sodium hydride, sodium alkoxide, sodium amide) to form an enolate intermediate, followed by treatment with an alkyl halide or sulfonate, is used for such transformations. Under certain conditions, the alkylation can be performed with an α,ω-dihaloalkane (such as 1,3-dibromopropane), such that a spiro linkage is formed. While Scheme 1 shows the formation of a spirocyclobutane (Compound II), other ring sizes (e.g., spirocyclopentane) are also accessible in this manner, by using other α,ω-dihaloalkanes. The carbonyl functionality can subsequently be converted into an exocyclic methylene (Compound III), using Wittig (or equivalent) chemistry. Treatment of exo-methylene compounds with hydrogen cyanide (or similar reagents, such as thiocyanates), in the presence of strong acid, can provide the corresponding tertiary formamido compounds (as in Compound IV), in a process known as the Ritter reaction. We have discovered that, under certain reaction conditions, both Compounds IV and V are formed, and that, under certain other reaction conditions, Compound V is the predominant product. Compound V presumably arises from a carbocation rearrangement process. Reduction of the formamido compounds, as a mixture (spiro and fused) or individually, using a hydride reducing agent, such as lithium aluminum hydride or sodium bis(methoxyethoxy)aluminum hydride, gives the corresponding secondary amines, Compounds VI and VII, respectively.

Another method for making compounds of the present invention utilizes Diels-Alder chemistry. Thus, as shown in Scheme 2, reaction of cyclopentadiene with cyclopentenyl dieneophiles (e.g., alkyl cyclopentene-1-carboxylates) will provide Diels-Alder adducts (Compounds X and VIII, respectively) that are readily transformed into compounds of the present invention. Such Diels-Alder chemistry is reported in the literature; see, for example, Deleens et al., Tetrahedron Lett. 43: 4963-4968 (2002) and U.S. Pat. No. 5,811,610. Conversion of Compound VIII into Compound IX can be accomplished by sequential reduction of the alkene (using catalytic hydrogenation conditions) and hydrolysis of the ester (using aqueous base). Similarly, conversion of Compound X into Compound XI can be accomplished by sequential reduction of the alkene (using catalytic hydrogenation conditions) and the nitro group (using tin or iron metal in aqueous hydrochloric acid). Alternately, both reductions could be accomplished simultaneously via catalytic hydrogenation. Compound IX can also be converted into Compound XI, as described by Koch and Haaf, Liebigs Ann. Chem. 638: 111-121 (1960). This reference also describes a synthesis of Compound IX from dicyclopentadiene. An alternate synthesis of Compound XI, through the intermediacy of the corresponding azide, is described by Zhdankin et al., J. Amer. Chem. Soc. 118: 5192-5197 (1996).

It will be appreciated by those of skill in the art of organic synthesis that the reactions described immediately above and in Scheme 2 are amenable to the inclusion of certain substituents. Thus, through the intermediacy of substituted versions of Compounds VIII, IX and X, substituted versions of Compound XI can be made. The most appropriate substituents are those which are compatible with both Diels-Alder chemistry and the subsequent chemistry leading to Compound XI. Those of skill in the art will appreciate the importance of the number and placement of such substituents, as the reactivity of the Diels-Alder reaction components (diene and dieneophile) can be greatly affected (positively or negatively) by the presence of such substituents. Thus, depending on where they are placed on either the diene component or the dieneophile component, such substituents include alkyl, alkoxy, aryloxy, alkoxycarbonyl (carboalkoxy), nitro and nitrile groups.

The Diels-Alder reaction is also amenable to the use of a variety of cyclic dienes and cyclic dieneophiles. Thus, the Diels-Alder adduct Compound XII (Scheme 2) can be made by reacting furan with an alkyl cyclopentene-1-carboxylate (similar to chemistry reported by Butler et al., Synlett 1:98-100 (2000)). Compound XIII can be made by reaction of 1,3-cyclohexadiene with 1-nitrocyclopentene or derivative thereof (similar to chemistry reported by Fuji et al., Tetrahedron: Asymmetry 3: 609-612 (1992)), and Compound XIV can be made by reacting cyclopentadiene with 1-nitrocyclohexene or derivative thereof (similar to chemistry reported by Deleens et al., Tetrahedron Lett. 43: 4963-4968 (2002)). Compounds XII, XIII and XIV can then be further transformed into compounds of the present invention, using chemistry described above or other similar chemistry.

Primary amines, such as Compound XI, can be converted into secondary amines through the intermediacy of amides and carbamates. Thus, sequential treatment of Compound XI with di-tert-butyl dicarbonate and lithium aluminum hydride will produce the corresponding N-methyl derivative. Such processes can also be use to convert secondary amines to tertiary amines. The present invention includes primary, secondary and tertiary amine compounds.

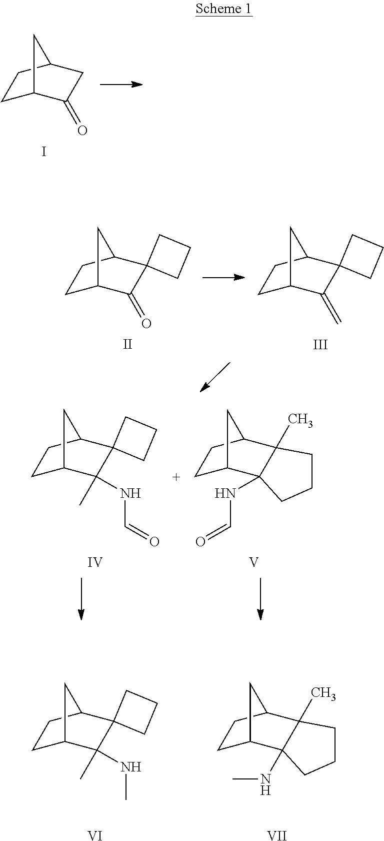

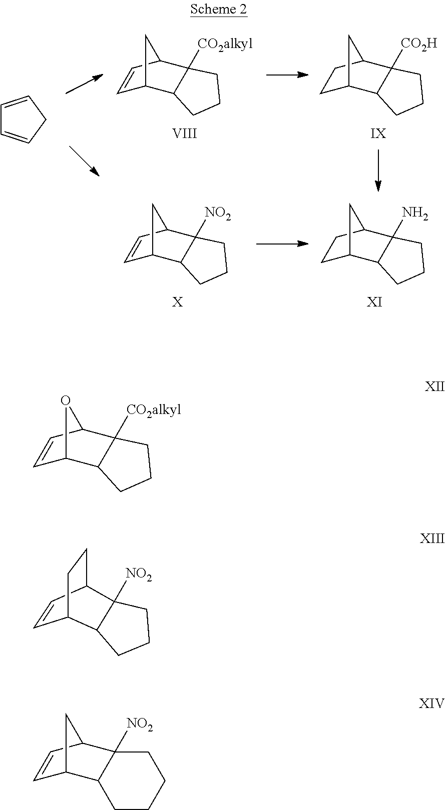

The incorporation of specific radioisotopes is also possible. For example, reductions of amides and carbamates with lithium aluminum deuteride or lithium aluminum tritide reducing agents can produce N-trideuteromethyl or N-tritritiomethyl amines. Alternatively, generation of an amide or carbamate, in which the carbonyl carbon is a ¹¹C, ¹³C, or ¹⁴C atom, followed by reduction with lithium aluminum hydride, will produce an amine with the ¹¹C, ¹³C, or ¹⁴C atom, respectively, incorporated. The incorporation of specific radioisotopes is often desirable in the preparation of compounds that are to be used in a diagnostic setting (e.g., as imaging agents) or in functional and metabolic studies.

### III. Pharmaceutical Compositions

Although it is possible to administer the compound of the present invention in the form of a bulk active chemical, it is preferred to administer the compound in the form of a pharmaceutical composition or formulation. Thus, one aspect the present invention includes pharmaceutical compositions comprising one or more compounds of Formula I and/or pharmaceutically acceptable salts thereof and one or more pharmaceutically acceptable carriers, diluents, or excipients. Another aspect of the invention provides a process for the preparation of a pharmaceutical composition including admixing one or more compounds of Formula I and/or pharmaceutically acceptable salts thereof with one or more pharmaceutically acceptable carriers, diluents or excipients.

The manner in which the compound of the present invention is administered can vary. The compound of the present invention is preferably administered orally. Preferred pharmaceutical compositions for oral administration include tablets, capsules, caplets, syrups, solutions, and suspensions. The pharmaceutical compositions of the present invention may be provided in modified release dosage forms such as time-release tablet and capsule formulations.

The pharmaceutical compositions can also be administered via injection, namely, intravenously, intramuscularly, subcutaneously, intraperitoneally, intraarterially, intrathecally, and intracerebroventricularly. Intravenous administration is a preferred method of injection. Suitable carriers for injection are well known to those of skill in the art and include 5% dextrose solutions, saline, and phosphate buffered saline.

The formulations may also be administered using other means, for example, rectal administration. Formulations useful for rectal administration, such as suppositories, are well known to those of skill in the art. The compounds can also be administered by inhalation, for example, in the form of an aerosol; topically, such as, in lotion form; transdermally, such as, using a transdermal patch (for example, by using technology that is commercially available from Novartis and Alza Corporation), by powder injection, or by buccal, sublingual, or intranasal absorption.

Pharmaceutical compositions may be formulated in unit dose form, or in multiple or subunit doses

The administration of the pharmaceutical compositions described herein can be intermittent, or at a gradual, continuous, constant or controlled rate. The pharmaceutical compositions may be administered to a warm-blooded animal, for example, a mammal such as a mouse, rat, cat, rabbit, dog, pig, cow, or monkey; but advantageously is administered to a human being. In addition, the time of day and the number of times per day that the pharmaceutical composition is administered can vary.

The compounds of the present invention may be used in the treatment of a variety of disorders and conditions and, as such, may be used in combination with a variety of other suitable therapeutic agents useful in the treatment or prophylaxis of those disorders or conditions. Thus, one embodiment of the present invention includes the administration of the compound of the present invention in combination with other therapeutic compounds. For example, the compound of the present invention can be used in combination with other NNR ligands (such as varenicline), allosteric modulators of NNRs, antioxidants (such as free radical scavenging agents), antibacterial agents (such as penicillin antibiotics), antiviral agents (such as nucleoside analogs, like zidovudine and acyclovir), anticoagulants (such as warfarin), anti-inflammatory agents (such as NSAIDs), anti-pyretics, analgesics, anesthetics (such as used in surgery), acetylcholinesterase inhibitors (such as donepezil and galantamine), antipsychotics (such as haloperidol, clozapine, olanzapine, and quetiapine), immuno-suppressants (such as cyclosporin and methotrexate), neuroprotective agents, steroids (such as steroid hormones), corticosteroids (such as dexamethasone, predisone, and hydrocortisone), vitamins, minerals, nutraceuticals, anti-depressants (such as imipramine, fluoxetine, paroxetine, escitalopram, sertraline, venlafaxine, and duloxetine), anxiolytics (such as alprazolam and buspirone), anticonvulsants (such as phenytoin and gabapentin), vasodilators (such as prazosin and sildenafil), mood stabilizers (such as valproate and aripiprazole), anti-cancer drugs (such as anti-proliferatives), antihypertensive agents (such as atenolol, clonidine, amlopidine, verapamil, and olmesartan), laxatives, stool softeners, diuretics (such as furosemide), anti-spasmotics (such as dicyclomine), anti-dyskinetic agents, and anti-ulcer medications (such as esomeprazole). Such a combination of pharmaceutically active agents may be administered together or separately and, when administered separately, administration may occur simultaneously or sequentially, in any order. The amounts of the compounds or agents and the relative timings of administration will be selected in order to achieve the desired therapeutic effect. The administration in combination of a compound of the present invention with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second. Such sequential administration may be close in time or remote in time.

Another aspect of the present invention includes combination therapy comprising administering to the subject a therapeutically or prophylactically effective amount of the compound of the present invention and one or more other therapy including chemotherapy, radiation therapy, gene therapy, or immunotherapy.

### IV. Method of Using Pharmaceutical Compositions

The compounds of the present invention can be used for the prevention or treatment of various conditions or disorders for which other types of nicotinic compounds have been proposed or are shown to be useful as therapeutics, such as CNS disorders, inflammation, inflammatory response associated with bacterial and/or viral infection, pain, metabolic syndrome, autoimmune disorders, addictions, obesity or other disorders described in further detail herein. This compound can also be used as a diagnostic agent (in vitro and in vivo). Such therapeutic and other teachings are described, for example, in references previously listed herein, including Williams et al., Drug News Perspec. 7(4): 205 (1994), Arneric et al., CNS Drug Rev. 1(1): 1-26 (1995), Arneric et al., Exp. Opin. Invest. Drugs 5(1): 79-100 (1996), Bencherif et al., J. Pharmacol. Exp. Ther. 279: 1413 (1996), Lippiello et al., J. Pharmacol. Exp. Ther. 279: 1422 (1996), Damaj et al., J. Pharmacol. Exp. Ther. 291: 390 (1999); Chiari et al., Anesthesiology 91: 1447 (1999), Lavand'homme and Eisenbach, Anesthesiology 91: 1455 (1999), Holladay et al., J. Med. Chem. 40(28): 4169-94 (1997), Bannon et al., Science 279: 77 (1998), PCT WO 94/08992, PCT WO 96/31475, PCT WO 96/40682, and U.S. Pat. No. 5,583,140 to Bencherif et al., U.S. Pat. No. 5,597,919 to Dull et al., U.S. Pat. No. 5,604,231 to Smith et al. and U.S. Pat. No. 5,852,041 to Cosford et al.

### CNS Disorders

The compounds and their pharmaceutical compositions are useful in the treatment or prevention of a variety of CNS disorders, including neurodegenerative disorders, neuropsychiatric disorders, neurologic disorders, and addictions. The compounds and their pharmaceutical compositions can be used to treat or prevent cognitive deficits and dysfunctions, age-related and otherwise; attentional disorders and dementias, including those due to infectious agents or metabolic disturbances; to provide neuroprotection; to treat convulsions and multiple cerebral infarcts; to treat mood disorders, compulsions and addictive behaviors; to provide analgesia; to control inflammation, such as mediated by cytokines and nuclear factor kappa B; to treat inflammatory disorders; to provide pain relief; and to treat infections, as anti-infectious agents for treating bacterial, fungal, and viral infections. Among the disorders, diseases and conditions that the compounds and pharmaceutical compositions of the present invention can be used to treat or prevent are: age-associated memory impairment (AAMI), mild cognitive impairment (MCI), age-related cognitive decline (ARCD), pre-senile dementia, early onset Alzheimer's disease, senile dementia, dementia of the Alzheimer's type, Alzheimer's disease, cognitive impairment no dementia (CIND), Lewy body dementia, HIV-dementia, AIDS dementia complex, vascular dementia, Down syndrome, head trauma, traumatic brain injury (TBI), dementia pugilistica, Creutzfeld-Jacob Disease and prion diseases, stroke, central ischemia, peripheral ischemia, attention deficit disorder, attention deficit hyperactivity disorder, dyslexia, schizophrenia, schizophreniform disorder, schizoaffective disorder, cognitive dysfunction in schizophrenia, cognitive deficits in schizophrenia, Parkinsonism including Parkinson's disease, postencephalitic parkinsonism, parkinsonism-dementia of Gaum, frontotemporal dementia Parkinson's Type (FTDP), Pick's disease, Niemann-Pick's Disease, Huntington's Disease, Huntington's chorea, dyskinesia, tardive dyskinesia, spastic dystonia, hyperkinesia, progressive supranuclear palsy, progressive supranuclear paresis, restless leg syndrome, Creutzfeld-Jakob disease, multiple sclerosis, amyotrophic lateral sclerosis (ALS), motor neuron diseases (MND), multiple system atrophy (MSA), corticobasal degeneration, Guillain-Barré Syndrome (GBS), and chronic inflammatory demyelinating polyneuropathy (CIDP), epilepsy, autosomal dominant nocturnal frontal lobe epilepsy, mania, anxiety, depression, including major depressive disorder (MDD), premenstrual dysphoria, panic disorders, bulimia, anorexia, narcolepsy, excessive daytime sleepiness, bipolar disorders, generalized anxiety disorder, obsessive compulsive disorder, rage outbursts, conduct disorder, oppositional defiant disorder, Tourette's syndrome, autism, drug and alcohol addiction, tobacco addiction and, thus, useful as an agent for smoking cessation, compulsive overeating and sexual dysfunction.

Cognitive impairments or dysfunctions may be associated with psychiatric disorders or conditions, such as schizophrenia and other psychotic disorders, including but not limited to psychotic disorder, schizophreniform disorder, schizoaffective disorder, delusional disorder, brief psychotic disorder, shared psychotic disorder, and psychotic disorders due to a general medical conditions, dementias and other cognitive disorders, including but not limited to mild cognitive impairment, pre-senile dementia, Alzheimer's disease, senile dementia, dementia of the Alzheimer's type, age-related memory impairment, Lewy body dementia, vascular dementia, AIDS dementia complex, dyslexia, Parkinsonism including Parkinson's disease, cognitive impairment and dementia of Parkinson's Disease, cognitive impairment of multiple sclerosis, cognitive impairment caused by traumatic brain injury, dementias due to other general medical conditions, anxiety disorders, including but not limited to panic disorder without agoraphobia, panic disorder with agoraphobia, agoraphobia without history of panic disorder, specific phobia, social phobia, obsessive-compulsive disorder, post-traumatic stress disorder, acute stress disorder, generalized anxiety disorder and generalized anxiety disorder due to a general medical condition, mood disorders, including but not limited to major depressive disorder, dysthymic disorder, bipolar depression, bipolar mania, bipolar I disorder, depression associated with manic, depressive or mixed episodes, bipolar II disorder, cyclothymic disorder, and mood disorders due to general medical conditions, sleep disorders, including but not limited to dyssomnia disorders, primary insomnia, primary hypersomnia, narcolepsy, parasomnia disorders, nightmare disorder, sleep terror disorder and sleepwalking disorder, mental retardation, learning disorders, motor skills disorders, communication disorders, pervasive developmental disorders, attention-deficit and disruptive behavior disorders, attention deficit disorder, attention deficit hyperactivity disorder, feeding and eating disorders of infancy, childhood, or adults, tic disorders, elimination disorders, substance-related disorders, including but not limited to substance dependence, substance abuse, substance intoxication, substance withdrawal, alcohol-related disorders, amphetamine or amphetamine-like-related disorders, caffeine-related disorders, cannabis-related disorders, cocaine-related disorders, hallucinogen-related disorders, inhalant-related disorders, nicotine-related disorders, opioid-related disorders, phencyclidine or phencyclidine-like-related disorders, and sedative-, hypnotic- or anxiolytic-related disorders, personality disorders, including but not limited to obsessive-compulsive personality disorder and impulse-control disorders.

Cognitive performance may be assessed with a validated cognitive scale, such as, for example, the cognitive subscale of the Alzheimer's Disease Assessment Scale (ADAS-cog). One measure of the effectiveness of the compounds of the present invention in improving cognition may include measuring a patient's degree of change according to such a scale.

Regarding compulsions and addictive behaviors, the compounds of the present invention may be used as a therapy for nicotine addiction, including as an agent for smoking cessation, and for other brain-reward disorders, such as substance abuse including alcohol addiction, illicit and prescription drug addiction, eating disorders, including obesity, and behavioral addictions, such as gambling, or other similar behavioral manifestations of addiction.

The above conditions and disorders are discussed in further detail, for example, in the American Psychiatric Association: Diagnostic and Statistical Manual of Mental Disorders, Fourth Edition, Text Revision, Washington, D.C., American Psychiatric Association, 2000. This Manual may also be referred to for greater detail on the symptoms and diagnostic features associated with substance use, abuse, and dependence.

### Inflammation

The nervous system, primarily through the vagus nerve, is known to regulate the magnitude of the innate immune response by inhibiting the release of macrophage tumor necrosis factor (TNF). This physiological mechanism is known as the “cholinergic anti-inflammatory pathway” (see, for example, Tracey, “The Inflammatory Reflex,” Nature 420: 853-9 (2002)). Excessive inflammation and tumor necrosis factor synthesis cause morbidity and even mortality in a variety of diseases. These diseases include, but are not limited to, endotoxemia, rheumatoid arthritis, osteoarthritis, psoriasis, asthma, atherosclerosis, idiopathic pulmonary fibrosis, and inflammatory bowel disease.

Inflammatory conditions that can be treated or prevented by administering the compounds described herein include, but are not limited to, chronic and acute inflammation, psoriasis, endotoxemia, gout, acute pseudogout, acute gouty arthritis, arthritis, rheumatoid arthritis, osteoarthritis, allograft rejection, chronic transplant rejection, asthma, atherosclerosis, mononuclear-phagocyte dependent lung injury, idiopathic pulmonary fibrosis, atopic dermatitis, chronic obstructive pulmonary disease, adult respiratory distress syndrome, acute chest syndrome in sickle cell disease, inflammatory bowel disease, irritable bowel syndrome, including diarrhea predominant IBS, Crohn's disease, ulcers, ulcerative colitis, acute cholangitis, aphthous stomatitis, cachexia, pouchitis, glomerulonephritis, lupus nephritis, thrombosis, and graft vs. host reaction.

Inflammatory Response Associated with Bacterial and/or Viral Infection

Many bacterial and/or viral infections are associated with side effects brought on by the formation of toxins, and the body's natural response to the bacteria or virus and/or the toxins. As discussed above, the body's response to infection often involves generating a significant amount of TNF and/or other cytokines. The over-expression of these cytokines can result in significant injury, such as septic shock (when the bacteria is sepsis), endotoxic shock, urosepsis, viral pneumonitis and toxic shock syndrome.

Cytokine expression is mediated by NNRs, and can be inhibited by administering agonists or partial agonists of these receptors. Those compounds described herein that are agonists or partial agonists of these receptors can therefore be used to minimize the inflammatory response associated with bacterial infection, as well as viral and fungal infections. Examples of such bacterial infections include anthrax, botulism, and sepsis. Some of these compounds may also have antimicrobial properties. Furthermore, the compounds can be used in the treatment of Raynaud's disease, namely viral-induced painful peripheral vasoconstriction.

These compounds can also be used as adjunct therapy in combination with existing therapies to manage bacterial, viral and fungal infections, such as antibiotics, antivirals and antifungals. Antitoxins can also be used to bind to toxins produced by the infectious agents and allow the bound toxins to pass through the body without generating an inflammatory response. Examples of antitoxins are disclosed, for example, in U.S. Pat. No. 6,310,043 to Bundle et al. Other agents effective against bacterial and other toxins can be effective and their therapeutic effect can be complemented by co-administration with the compounds described herein.

### Pain

The compounds can be administered to treat and/or prevent pain, including acute, neurologic, inflammatory, neuropathic and chronic pain. The compounds can be used in conjunction with opiates to minimize the likelihood of opiate addiction (e.g., morphine sparing therapy). The analgesic activity of compounds described herein can be demonstrated in models of persistent inflammatory pain and of neuropathic pain, performed as described in U.S. Published Patent Application No. 20010056084 A1 (Allgeier et al.) (e.g., mechanical hyperalgesia in the complete Freund's adjuvant rat model of inflammatory pain and mechanical hyperalgesia in the mouse partial sciatic nerve ligation model of neuropathic pain).

The analgesic effect is suitable for treating pain of various genesis or etiology, in particular in treating inflammatory pain and associated hyperalgesia, neuropathic pain and associated hyperalgesia, chronic pain (e.g., severe chronic pain, post-operative pain and pain associated with various conditions including cancer, angina, renal or biliary colic, menstruation, migraine, and gout). Inflammatory pain may be of diverse genesis, including arthritis and rheumatoid disease, teno-synovitis and vasculitis. Neuropathic pain includes trigeminal or herpetic neuralgia, neuropathies such as diabetic neuropathy pain, causalgia, low back pain and deafferentation syndromes such as brachial plexus avulsion.

### Neovascularization

Inhibition of neovascularization, for example, by administering antagonists (or at certain dosages, partial agonists) of nicotinic receptors can treat or prevent conditions characterized by undesirable neovascularization or angiogenesis. Such conditions can include those characterized by inflammatory angiogenesis and/or ischemia-induced angiogenesis. Neovascularization associated with tumor growth can also be inhibited by administering those compounds described herein that function as antagonists or partial agonists of nicotinic receptors.

Specific antagonism of nicotinic receptors reduces the angiogenic response to inflammation, ischemia, and neoplasia. Guidance regarding appropriate animal model systems for evaluating the compounds described herein can be found, for example, in Heeschen, C. et al., “A novel angiogenic pathway mediated by non-neuronal nicotinic acetylcholine receptors,” J. Clin. Invest. 110(4):527-36 (2002).

Representative tumor types that can be treated using the compounds described herein include SCLC, NSCLC, ovarian cancer, pancreatic cancer, breast carcinoma, colon carcinoma, rectum carcinoma, lung carcinoma, oropharynx carcinoma, hypopharynx carcinoma, esophagus carcinoma, stomach carcinoma, pancreas carcinoma, liver carcinoma, gallbladder carcinoma, bile duct carcinoma, small intestine carcinoma, urinary tract carcinoma, kidney carcinoma, bladder carcinoma, urothelium carcinoma, female genital tract carcinoma, cervix carcinoma, uterus carcinoma, ovarian carcinoma, choriocarcinoma, gestational trophoblastic disease, male genital tract carcinoma, prostate carcinoma, seminal vesicles carcinoma, testes carcinoma, germ cell tumors, endocrine gland carcinoma, thyroid carcinoma, adrenal carcinoma, pituitary gland carcinoma, skin carcinoma, hemangiomas, melanomas, sarcomas, bone and soft tissue sarcoma, Kaposi's sarcoma, tumors of the brain, tumors of the nerves, tumors of the eyes, tumors of the meninges, astrocytomas, gliomas, glioblastomas, retinoblastomas, neuromas, neuroblastomas, Schwannomas, meningiomas, solid tumors arising from hematopoietic malignancies (such as leukemias, chloromas, plasmacytomas and the plaques and tumors of mycosis fungoides and cutaneous T-cell lymphoma/leukemia), and solid tumors arising from lymphomas.

The compounds can also be administered in conjunction with other forms of anti-cancer treatment, including co-administration with antineoplastic antitumor agents such as cis-platin, adriamycin, daunomycin, and the like, and/or anti-VEGF (vascular endothelial growth factor) agents, as such are known in the art.

The compounds can be administered in such a manner that they are targeted to the tumor site. For example, the compounds can be administered in microspheres, microparticles or liposomes conjugated to various antibodies that direct the microparticles to the tumor. Additionally, the compounds can be present in microspheres, microparticles or liposomes that are appropriately sized to pass through the arteries and veins, but lodge in capillary beds surrounding tumors and administer the compounds locally to the tumor. Such drug delivery devices are known in the art.

### Other Disorders

In addition to treating CNS disorders, inflammation, and neovascularization, and pain, the compounds of the present invention can be also used to prevent or treat certain other conditions, diseases, and disorders in which NNRs play a role. Examples include autoimmune disorders such as lupus, disorders associated with cytokine release, cachexia secondary to infection (e.g., as occurs in AIDS, AIDS related complex and neoplasia), obesity, pemphitis, urinary incontinence, overactive bladder (OAB), diarrhea, constipation, retinal diseases, infectious diseases, myasthenia, Eaton-Lambert syndrome, hypertension, preeclampsia, osteoporosis, vasoconstriction, vasodilatation, cardiac arrhythmias, type I diabetes, type II diabetes, bulimia, anorexia and sexual dysfunction, as well as those indications set forth in published PCT application WO 98/25619. The compounds of this invention can also be administered to treat convulsions such as those that are symptomatic of epilepsy, and to treat conditions such as syphillis and Creutzfeld-Jakob disease.

Compounds of the present invention may be used to treat bacterial infections and dermatologic conditions, such as pemphigus folliaceus, pemphigus vulgaris, and other disorders, such as acantholysis, where autoimmune responses with high ganglionic NNR antibody titer is present. In these disorders, and in other autoimmune diseases, such as Mysthenia Gravis, the fab fragment of the antibody binds to the NNR receptor (crosslinking 2 receptors), which induces internalization and degradation.

### Diagnostic Uses

The compounds can be used in diagnostic compositions, such as probes, particularly when they are modified to include appropriate labels. For this purpose the compounds of the present invention most preferably are labeled with the radioactive isotopic moiety ¹¹C.

The administered compounds can be detected using position emission topography (PET). A high specific activity is desired to visualize the selected receptor subtypes at non-saturating concentrations. The administered doses typically are below the toxic range and provide high contrast images. The compounds are expected to be capable of administration in non-toxic levels. Determination of dose is carried out in a manner known to one skilled in the art of radiolabel imaging. See, for example, U.S. Pat. No. 5,969,144 to London et al.

The compounds can be administered using known techniques. See, for example, U.S. Pat. No. 5,969,144 to London et al., as noted. The compounds can be administered in formulation compositions that incorporate other ingredients, such as those types of ingredients that are useful in formulating a diagnostic composition. Compounds useful in accordance with carrying out the present invention most preferably are employed in forms of high purity. See, U.S. Pat. No. 5,853,696 to Elmalch et al.

After the compounds are administered to a subject (e.g., a human subject), the presence of that compound within the subject can be imaged and quantified by appropriate techniques in order to indicate the presence, quantity, and functionality. In addition to humans, the compounds can also be administered to animals, such as mice, rats, dogs, and monkeys. SPECT and PET imaging can be carried out using any appropriate technique and apparatus. See Villemagne et al., In: Arneric et al. (Eds.) Neuronal Nicotinic Receptors: Pharmacology and Therapeutic Opportunities, 235-250 (1998) and U.S. Pat. No. 5,853,696 to Elmalch et al., each herein incorporated by reference, for a disclosure of representative imaging techniques.

### V. Synthetic Examples

### Example 1

### 3-Ethylspiro[bicyclo[2.2.1]heptane-2,1′-cyclobutan]-3-ol and spiro[bicyclo[2.2.1]heptane-2,1-cyclobutan]-3-ol

To a solution of 2-norbornanone (norcamphor) (74.0 g, 0.673 mol) and 1,3-dibromopropane (190 g, 0.942 mol) in diethyl ether (2.2 L) was added sodium amide (65.6 g, 1.68 mol), and the mixture was stirred at reflux for 24 h. The reaction was incomplete, by GCMS analysis. An additional 0.1 equivalent (13.6 g, 67.3 mmol) of 1,3-dibromopropane and 0.5 equivalent (13.0 g, 0.336 mol) of sodium amide were added, and the mixture was stirred at reflux for another 24 h period. The reaction was still not complete, so the cycle of additional of reagents, stirring at reflux and GCMS analysis was repeated three more times, resulting in the addition of another 0.15 equivalents of 1,3-dibromopropane and another 3.5 equivalents of sodium amide over a period of ˜40 h at reflux. Finally, GCMS analysis indicated that starting material had disappeared. The reaction was cooled to −10° C. and slowly quenched (stirring) with water (600 mL). The stirring was stopped, and the layers separated. The organic layer was washed successively with 1 M aqueous hydrochloric acid (100 mL), water (50 mL) and saturated aqueous sodium chloride (50 mL). The organic layer was then combined with water (1500 mL) and stirred vigorously as solid potassium permanganate (341 g) was added, in portions, over an 8 h period. The mixture was then stirred for 2 days at ambient temperature and filtered through diatomaceous earth. The organic layer was separated, and the aqueous layer was extracted with ether (2×500 mL). The organic layers were combined, washed with saturated aqueous sodium chloride (50 mL), and dried over anhydrous sodium sulfate. The solvent was evaporated, and the crude product (85 g) was purified on a silica gel column. Selected fractions were concentrated to give spiro[bicyclo[2.2.1]heptane-2,1′-cyclobutan]-3-one (29 g, 29% yield). ¹H NMR (CDCl₃, 400 MHz): δ 2.55-2.49 (m, 2H), 2.18-2.08 (m, 2H), 2.00-1.58 (m, 7H), 1.49-1.36 (m, 3H); LCMS (m/z): 151 (M+1).

A solution of spiro[bicyclo[2.2.1]heptane-2,1′-cyclobutan]-3-one (27.5 g, 0.183 mol) in tetrahydrofuran (THF) (500 mL) was cooled to 0° C. and a solution of 3 M ethylmagnesium bromide in ether (122 mL, 0.366 mol) was added drop-wise, at such a rate that the internal temperature of the reaction mixture was maintained below 5° C. (20 min addition time). The resulting solution was stirred at 2-5° C. for 30 min and then stirred at ambient temperature for 20 h. The reaction was cooled to −10° C. and water (100 mL) was added to quench the reaction. Additional water (300 mL) and ethyl acetate (300 mL) were then added, and the mixture was stirred. The stirring was stopped, and the organic layer was separated and concentrated. The residue from the organic layer was combined with the aqueous layer and extracted with ethyl acetate (3×400 mL). The ethyl acetate extracts were combined and washed with saturated aqueous sodium chloride (50 mL) and dried over anhydrous sodium sulfate. The solvent was evaporated, and crude material was purified on a silica gel column, eluting with 0-10% ethyl acetate in hexanes, to afford 3-ethylspiro[bicyclo[2.2.1]heptane-2,1′-cyclobutan]-3-ol (16.2 g, 47%) and spiro[bicyclo[2.2.1]heptane-2,1′-cyclobutan]-3-ol (13.2 g, 46%), as colorless oils. These materials were used without further purification is subsequent syntheses.

### Example 2

### (3aS,4S,7R,7aS)-7a-Ethyl-N-methyloctahydro-4,7-methano-1H-inden-3a-amine hydrochloride and (3aR,4R,7S,7aR)-7a-ethyl-N-methyloctahydro-4,7-methano-1H-inden-3a-amine hydrochloride

A 500 mL one-neck flask was charged with 3-ethylspiro[bicyclo[2.2.1]heptane-2,1′-cyclobutan]-3-ol (15.3 g, 85.0 mmol) and sodium cyanide (8.33 g, 0.170 mol) and sealed with a rubber septum. A needle, with a balloon attached, was inserted into the septum. Acetic acid (28.2 mL, 0.493 mol) was added by syringe, and the mixture was stirred for 5 min at ambient temperature. The mixture was then cooled to 0° C., and concentrated sulfuric acid (28.6 mL, 0.536 mol) was added drop-wise by syringe, over a 40 min period. The resulting solution was stirred at 0° C. for 30 min and then at ambient temperature for 16 h. The reaction was cooled to −10° C. and water (50 mL) was added to quench the reaction. Chloroform (50 mL) was then added, followed by 10 M aqueous sodium hydroxide (200 mL, 2.0 mol). The resulting mixture had a pH of 12. The mixture was transferred to a separatory funnel, combined with water (600 mL), and extracted with chloroform (3×500 mL). The organic layers were combined, washed with saturated aqueous sodium chloride (50 mL) and dried over anhydrous sodium sulfate. The solvent was evaporated, and the crude product was purified on a silica gel column, eluting with 10-50% ethyl acetate in hexanes, to afford N-(7a-ethyloctahydro-4,7-methano-1H-inden-3a-yl)formamide as white solid (10.2 g, 56% yield).

To a flask containing of anhydrous THF (140 mL) was added a solution of 1 M lithium aluminum hydride in THF (138 mL, 0.138 mol). The lithium aluminum hydride solution was heated to reflux, and solid N-(7a-ethyloctahydro-4,7-methano-1H-inden-3a-yl)formamide (9.5 g, 45.9 mmol) was added in portions over a 15 min period. The resulting mixture was refluxed for 21 h, cooled to −10° C. and quenched by slow addition of 5 M aqueous sodium hydroxide (18 mL). The resulting mixture was filtered through diatomaceous earth and washed with THF (3×150 mL). The filtrate was concentrated, and the residue was purified on two silica gel columns. The first was eluted with 10-50% ethyl acetate in hexanes. Selected fractions were concentrated, and the residue was then applied to the second column, which was eluted with dichloromethane/methanol/aqueous ammonia (from 9:1:0.1 to 8:2:0.2) (v/v). Concentration of selected fractions gave 7a-ethyl-N-methyloctahydro-4,7-methano-1H-inden-3a-amine (8.2 g, 93% yield). ¹H NMR (CD₃OD, 400 MHz): δ 2.81 (s, 3H), 2.52 (d, J=3.2 Hz, 1H), 2.27 (brs, 1H), 2.23-2.16 (m, 1H), 2.06-2.02 (m, 1H), 1.88-1.52 (m, 9H), 1.44-1.22 (m, 3H), 1.07 (t, J=7.0 Hz, 3H); LCMS (m/z): 194 (M+1).

Racemic 7a-ethyl-N-methyloctahydro-4,7-methano-1H-inden-3a-amine (2.0 g) was dissolved in acetonitrile (10 mL) and was separated by chiral HPLC, using a ChiralPak AD-H, 5 micron, 250×20 cm column and eluting with 0.2% diethylamine, 5% isopropanol in acetonitrile (0.25 mL injections), with a flow rate of 10 mL/min. Selected fractions for each of the two peaks were concentrated and dissolved in 2 mL of methanol. Each of the two methanol solutions was treated with 10 mL of 1 M aqueous hydrochloric acid at ambient temperature. The resulting reaction mixtures were concentrated in a vacuum centrifuge, providing (3aS,4S,7R,7aS)-7a-ethyl-N-methyloctahydro-4,7-methano-1H-inden-3a-amine hydrochloride as the early eluting enantiomer (0.845 g, 35% recovery) and (3aR,4R,7S,7aR)-7a-ethyl-N-methyloctahydro-4,7-methano-1H-inden-3a-amine hydrochloride as late eluting enantiomer (0.630 g, 26% recovery), as white powders.

(3aS,4S,7R,7aS)-7a-ethyl-N-methyloctahydro-4,7-methano-1H-inden-3a-amine hydrochloride: ¹H NMR (D₂O, 400 MHz): δ 2.80 (s, 3H), 2.51 (d, J=3.2 Hz, 1H), 2.26 (brs, 1H), 2.23-2.16 (m, 1H), 2.06-2.02 (m, 1H), 1.88-1.52 (m, 9H), 1.44-1.22 (m, 3H), 1.06 (t, J=7.1 Hz, 3H); LCMS (m/z): 194 (M+1).

(3aR,4R,7S,7aR)-7a-ethyl-N-methyloctahydro-4,7-methano-1H-inden-3a-amine hydrochloride: ¹H NMR (D₂O, 400 MHz): δ 2.81 (s, 3H), 2.52 (d, J=3.5 Hz, 1H), 2.27 (brs, 1H), 2.23-2.16 (m, 1H), 2.06-2.02 (m, 1H), 1.88-1.52 (m, 9H), 1.44-1.22 (m, 3H), 1.06 (t, J=7.1 Hz, 3H); LCMS (m/z): 194 (M+1).

### Example 3

### (3aS,4S,7R,7aS)-N-Methyloctahydro-4,7-methano-1H-inden-3a-amine hydrochloride and (3aR,4R,7S,7aR)-N-methyloctahydro-4,7-methano-1H-inden-3a-amine hydrochloride

A 500 mL one-neck flask was charged with spiro[bicyclo[2.2.1]heptane-2,1′-cyclobutan]-3-ol (12.3 g, 80.9 mmol) and sodium cyanide (6.70 g, 0.137 mol) and sealed with a rubber septum. A needle, with a balloon attached, was inserted into the septum. Acetic acid (22.7 mL, 0.396 mol) was added by syringe, and the mixture was stirred for 5 min at ambient temperature. The mixture was then cooled to 0° C., and concentrated sulfuric acid (23.0 mL, 0.430 mol) was added drop-wise by syringe, over a 30 min period. The resulting solution was stirred at 0° C. for 30 min and then at ambient temperature for 16 h. The reaction was cooled to −10° C. and water (50 mL) was added to quench the reaction. Chloroform (50 mL) was then added, followed by 10 M aqueous sodium hydroxide (170 mL, 1.7 mol). The resulting mixture had a pH of 12. The mixture was transferred to a separatory funnel, combined with water (400 mL), and extracted with chloroform (3×400 mL). The organic layers were combined, washed with saturated aqueous sodium chloride (50 mL) and dried over anhydrous sodium sulfate. The solvent was evaporated, and the crude product was purified on a silica gel column, eluting with 10-50% ethyl acetate in hexanes, to afford N-(octahydro-4,7-methano-1H-inden-3a-yl)formamide as white solid (10.7 g, 74% yield).

To a flask containing of anhydrous THF (170 mL) was added a solution of 1 M lithium aluminum hydride in THF (168 mL, 0.168 mol). The lithium aluminum hydride solution was heated to reflux, and solid N-(octahydro-4,7-methano-1H-inden-3a-yl)formamide (10.0 g, 55.9 mmol) was added in portions over a 15 min period. The resulting mixture was refluxed for 22 h, cooled to −10° C. and quenched by slow addition of 5 M aqueous sodium hydroxide (20 mL). The resulting mixture was filtered through diatomaceous earth and washed with THF (3×300 mL). The filtrate was concentrated, and the residue was purified on two silica gel columns. The first was eluted with 10-50% ethyl acetate in hexanes. Selected fractions were concentrated, and the residue was then applied to the second column, which was eluted with dichloromethane/methanol/aqueous ammonia (from 9:1:0.1 to 8:2:0.2) (v/v).

Concentration of selected fractions gave N-methyloctahydro-4,7-methano-1H-inden-3a-amine (8.1 g, 88% yield). ¹H NMR (D₂O, 400 MHz): δ 2.80 (s, 3H), 2.54 (brs, 1H), 2.27-2.16 (m, 3H), 1.95-1.56 (m, 7H), 1.49-1.32 (m, 4H); LCMS (m/z): 166 (M+1).

Racemic N-methyloctahydro-4,7-methano-1H-inden-3a-amine (1.5 g) was dissolved in acetonitrile (15 mL) and was separated by chiral HPLC, using a ChiralPak AD, 5 micron, 250×20 cm column and eluting with 0.2% diethylamine, 5% isopropanol in acetonitrile (0.25 mL injections), with a flow rate of 10 mL/min. Selected fractions for each of the two peaks were concentrated and dissolved in 2 mL of methanol. Each of the two methanol solutions was treated with 2 mL of 2 M aqueous hydrochloric acid at ambient temperature. The resulting reaction mixtures were concentrated in a vacuum centrifuge, providing (3aS,4S,7R,7aS)-N-methyloctahydro-4,7-methano-1H-inden-3a-amine hydrochloride (0.460 g, 26% recovery) as early eluting enantiomer and (3aR,4R,7S,7aR)-N-methyloctahydro-4,7-methano-1H-inden-3a-amine hydrochloride (0.480 g, 27% recovery) as late eluting enantiomer, as white powders.

(3aS,4S,7R,7aS)-N-methyloctahydro-4,7-methano-1H-inden-3a-amine hydrochloride: ¹H NMR (D₂O, 400 MHz): δ 2.81 (s, 3H), 2.55 (brs, 1H), 2.27-2.16 (m, 3H), 1.95-1.56 (m, 7H), 1.49-1.32 (m, 4H); LCMS (m/z): 166 (M+1).

(3aR,4R,7S,7aR)-N-methyloctahydro-4,7-methano-1H-inden-3a-amine hydrochloride: ¹H NMR (D₂O, 400 MHz): δ 2.81 (s, 3H), 2.55 (brs, 1H), 2.27-2.16 (m, 3H), 1.95-1.56 (m, 7H), 1.49-1.32 (m, 4H); LCMS (m/z): 166 (M+1).

### Example 4

### (3aS,4S,7R,7aS)-N,N,N-Trimethyloctahydro-4,7-methano-1H-inden-3a-ammonium formate

A mixture of (3aS,4S,7R,7aS)-N-methyloctahydro-4,7-methano-1H-inden-3a-amine (30 mg, 0.18 mmol), iodomethane (2.0 mL, 32 mmol) and potassium carbonate (1.0 g, 7.2 mmol) in THF (2 mL) was placed in pressure tube and stirred at 100° C. for 48 h. The reaction mixture was filtered, and the filtrate was concentrated. The residue was purified by HPLC, eluting with mixtures of 0.05% aqueous formic acid and 0.05% formic acid in acetonitrile. Selected fractions were combined and concentrated to obtain (3aS,4S,7R,7aS)-N,N,N-trimethyloctahydro-4,7-methano-1H-inden-3a-ammonium formate (20 mg).

¹H NMR (CD₃OD, 400 MHz): δ 8.52 (brs, 1H), 3.18 (s, 9H), 2.67 (s, 1H), 2.58-2.45 (m, 2H), 2.35-2.24 (m, 1H), 2.17-2.12 (m, 1H), 1.98-1.62 (m, 7H), 1.58-1.36 (m, 3H); LCMS (m/z): 194 (M).

### Example 5

### (3aR,4R,7S,7aR)-N,N,N-Trimethyloctahydro-4,7-methano-1H-inden-3a-ammonium formate

A mixture of (3aR,4R,7S,7aR)-N-methyloctahydro-4,7-methano-1H-inden-3a-amine (30 mg, 0.18 mmol), iodomethane (2.0 mL, 32 mmol) and potassium carbonate (1.0 g, 7.2 mmol) in THF (2 mL) was placed in pressure tube and stirred at 100° C. for 48 h. The reaction mixture was filtered, and the filtrate was concentrated. The residue was purified by HPLC, eluting with mixtures of 0.05% aqueous formic acid and 0.05% formic acid in acetonitrile. Selected fractions were combined and concentrated to obtain (3aR,4R,7S,7aR)-N,N,N-trimethyloctahydro-4,7-methano-1H-inden-3a-ammonium formate (20 mg).

¹H NMR (CD₃OD, 400 MHz): δ 8.51 (brs, 1H), 3.18 (s, 9H), 2.67 (s, 1H), 2.58-2.45 (m, 2H), 2.35-2.24 (m, 1H), 2.17-2.12 (m, 1H), 1.98-1.62 (m, 7H), 1.58-1.36 (m, 3H); LCMS (m/z): 194 (M).

### Example 6

### (3aS,4S,7R,7aS)-N,N-Dimethyloctahydro-4,7-methano-1H-inden-3a-amine hydroiodide

Iodomethane (1.0 mL, 16 mmol) was added to a solution of (3aS,4S,7R,7aS)-N-methyloctahydro-4,7-methano-1H-inden-3a-amine (0.11 g, 0.67 mmol) in acetonitrile (3 mL), and the mixture was stirred at ambient temperature for 18 h. Anhydrous ether (30 mL) was added to the reaction, and the mixture was centrifuged. The supernatant was decanted, and the remaining solid was dried to obtain (3aS,4S,7R,7aS)-N,N-dimethyloctahydro-4,7-methano-1H-inden-3a-amine hydroiodide (0.19 g, 93% yield) as off-white solid. ¹H NMR (CD₃OD, 400 MHz): δ 2.92 (s, 3H), 2.88 (s, 3H), 2.53 (brs, 1H), 2.45-2.25 (m, 2H), 2.15 (d, J=3.5 Hz, 1H), 2.10-2.06 (m, 1H), 1.96-1.65 (m, 6H), 1.55-1.38 (m, 4H); LCMS (m/z): 180 (M+1).

### Example 7

### (3aR,4R,7S,7aR)-N,N-Dimethyloctahydro-4,7-methano-1H-inden-3a-amine hydroiodide

Iodomethane (1.0 mL, 16 mmol) was added to a solution of (3aR,4R,7S,7aR)-N-methyloctahydro-4,7-methano-1H-inden-3a-amine (0.11 g, 0.67 mmol) in acetonitrile (3 mL), and the mixture was stirred at ambient temperature for 18 h. Anhydrous ether (30 mL) was added to the reaction, and the mixture was centrifuged. The supernatant was decanted, and the remaining solid was dried to obtain (3aR,4R,7S,7aR)-N,N-dimethyloctahydro-4,7-methano-1H-inden-3a-amine hydroiodide (0.185 g, 90% yield) as off-white solid. ¹H NMR (CD₃OD, 400 MHz): δ 2.92 (s, 3H), 2.88 (s, 3H), 2.53 (brs, 1H), 2.45-2.25 (m, 2H), 2.15 (d, J=3.5 Hz, 1H), 2.10-2.06 (m, 1H), 1.96-1.65 (m, 6H), 1.55-1.38 (m, 4H); LCMS (m/z): 180 (M+1).

### Example 8

### (3aS,4S,7R,7aS)-7a-Ethyl-N,N-dimethyloctahydro-4,7-methano-1H-inden-3a-amine trifluoroacetate salt

Iodomethane (1.0 mL, 16 mmol) was added to a solution of (3aS,4S,7R,7aS)-7a-ethyl-N-methyloctahydro-4,7-methano-1H-inden-3a-amine (0.10 g, 0.52 mmol) in acetonitrile (3 mL), and the mixture was stirred at ambient temperature for 18 h. The reaction was concentrated and purified by HPLC, eluting with mixtures of 0.05% TFA in water and 0.05% TFA in acetonitrile. Selected fractions were concentrated to obtain (3aS,4S,7R,7aS)-7a-ethyl-N,N-dimethyloctahydro-4,7-methano-1H-inden-3a-amine trifluoroacetate salt (20 mg). ¹H NMR (CD₃OD, 400 MHz): δ 2.93 (s, 3H), 2.91 (s, 3H), 2.48-2.40 (m, 2H), 2.19 (brs, 1H), 2.05-1.95 (m, 1H), 1.88-1.81 (m, 2H), 1.76-1.52 (m, 7H), 1.49-1.42 (m, 1H), 1.38-1.32 (m, 2H), 1.06 (t, J=7.1 Hz, 3H); LCMS (m/z): 208 (M+1).

### Example 9

### (3aR,4R,7S,7aR)-7a-Ethyl-N,N-dimethyloctahydro-4,7-methano-1H-inden-3a-amine trifluoroacetate

Iodomethane (1.0 mL, 16 mmol) was added to a solution of (3aR,4R,7S,7aR)-7a-ethyl-N-methyloctahydro-4,7-methano-1H-inden-3a-amine (0.10 g, 0.52 mmol) in acetonitrile (3 mL), and the mixture was stirred at ambient temperature for 18 h. The reaction was concentrated and purified by HPLC, eluting with mixtures of 0.05% TFA in water and 0.05% TFA in acetonitrile. Selected fractions were concentrated to obtain (3aR,4R,7S,7aR)-7a-ethyl-N,N-dimethyloctahydro-4,7-methano-1H-inden-3a-amine trifluoroacetate salt (16 mg). ¹H NMR (CD₃OD, 400 MHz): δ 2.93 (s, 3H), 2.91 (s, 3H), 2.48-2.40 (m, 2H), 2.19 (brs, 1H), 2.05-1.95 (m, 1H), 1.88-1.81 (m, 2H), 1.76-1.52 (m, 7H), 1.49-1.42 (m, 1H), 1.38-1.32 (m, 2H), 1.06 (t, J=7.1 Hz, 3H); LCMS (m/z): 208 (M+1).

### Example 10

### N-Methyl-7a-propyloctahydro-4,7-methano-1H-inden-3a-amine hydrochloride

A solution of spiro[bicyclo[2.2.1]heptane-2,1′-cyclobutan]-3-one (500 mg, 3.33 mmol) in THF (10 mL) was cooled to 0° C. and a solution of 2.0 M n-propylmagnesium chloride in ether (5.0 mL, 10 mmol) was added drop-wise. The resulting solution was warmed slowly to ambient temperature and then stirred at ambient temperature for 20 h. The reaction was quenched by addition of saturated aqueous ammonium chloride (5 mL), concentrated. The residue was partitioned between water (30 mL) and dichloromethane (20 mL). The organic layer was dried over anhydrous sodium sulfate and concentrated to obtain 500 mg of 3-propylspiro[bicyclo[2.2.1]heptane-2,1′-cyclobutan]-3-ol (˜60% pure by GCMS analysis).

The 3-propylspiro[bicyclo[2.2.1]heptane-2,1′-cyclobutan]-3-ol was dissolved in acetic acid (1.0 mL, 17 mmol) combined with sodium cyanide (185 mg, 3.62 mmol), and cooled in an ice bath. Sulfuric acid (1.0 mL, 19 mmol) was slowly added drop-wise, and then the reaction mixture was slowly warmed to ambient temperature, at which temperature it was stirred for 18 h. The reaction mixture was diluted with water (20 mL) and extracted with dichloromethane (30 mL). The organic layer was washed with 10% aqueous sodium hydroxide (20 mL), dried over anhydrous sodium sulfate and concentrated. The residue was dissolved in THF (30 mL), cooled in an ice bath, and 1.0 M lithium aluminum hydride in THF (3.1 mL, 3.1 mmol) was slowly added. The reaction was then heated at reflux for 17 h, cooled in an ice bath, and slowly quenched with solid sodium sulfate decahydrate (5 g). The mixture was filtered, and the filtrated was concentrated. The residue was purified by HPLC, eluting with mixtures of 0.05% formic acid in water and 0.05% formic acid in acetonitrile. Product containing fractions were combined, made basic (to pH 9) by addition of 3 M aqueous sodium hydroxide and extracted with dichloromethane (30 mL). Aqueous hydrochloric acid (2 mL of 2.0 M) was added to the dichloromethane extract, and the mixture was concentrated and vacuum dried, leaving N-methyl-7a-propyloctahydro-4,7-methano-1H-inden-3a-amine hydrochloride (120 mg) as white solid. ¹H NMR (D₂O, 400 MHz): δ 2.81 (s, 3H), 2.51 (d, J=2.8 Hz, 1H), 2.27-2.16 (m, 2H), 2.06-2.01 (m, 1H), 1.86-1.32 (m, 13H), 1.23-1.15 (m, 1H), 1.04 (t, J=7.0 Hz, 3H); LCMS (m/z): 208 (M+1).

### Example 11

### 7a-Butyl-N-methyloctahydro-4,7-methano-1H-inden-3a-amine hydrochloride

To a solution of spiro[bicyclo[2.2.1]heptane-2,1′-cyclobutan]-3-one (4.0 g, 27 mmol) in THF (50 mL) at −78° C. was slowly added n-butyllithium (16 mL of 2.5 M in hexanes, 40 mmol). The reaction was slowly warmed to ambient temperature (over a period of 4 h), slowly quenched with saturated aqueous ammonium chloride (20 mL), and concentrated. The residue was partitioned between dichloromethane (100 mL) and water (50 mL). The organic layer was dried over anhydrous sodium sulfate and concentrated to obtain 3-butylspiro[bicyclo[2.2.1]heptane-2,1′-cyclobutan]-3-ol (5.5 g) as oil. To a solution of 3-butylspiro[bicyclo[2.2.1]heptane-2,1′-cyclobutan]-3-ol (5.5 g, 26 mmol) in acetic acid (11.0 mL, 192 mmol) was added sodium cyanide (2.02 g, 39.6 mmol), and the mixture was cooled in an ice bath. Sulfuric acid (12.0 mL, 225 mmol) was slowly added drop-wise, and then the reaction was slowly warmed to ambient temperature, at which temperature it was stirred for 18 h. The reaction was diluted with water (200 mL) and extracted with dichloromethane (100 mL). The organic layer was washed with 10% aqueous sodium hydroxide (50 mL), dried over anhydrous sodium sulfate and concentrated. The residue was dissolved in THF (60 mL), cooled in an ice bath, and treated drop-wise with 1.0 M lithium aluminum hydride in THF (53 mL, 53 mmol). The reaction was then refluxed for 17 h, cooled in an ice bath, and slowly quenched with solid sodium sulfate decahydrate (20 g). This mixture was filtered, and the filtrated was concentrated. The residue was purified on a silica gel column, eluting with mixtures of chloroform/methanol/aqueous ammonia (9:1.0:0.1) in chloroform, to obtain 7a-butyl-N-methyloctahydro-4,7-methano-1H-inden-3a-amine (4.74 g, 79% yield) as oil. To a solution of 7a-butyl-N-methyloctahydro-4,7-methano-1H-inden-3a-amine (0.12 g, 0.54 mmol) in dichloromethane (2 mL) at 0° C. was added concentrated hydrochloric acid (0.1 mL). The mixture was concentrated and vacuum dried to obtain 7a-butyl-N-methyloctahydro-4,7-methano-1H-inden-3a-amine hydrochloride (0.11 g) as white solid.

¹H NMR (D₂O, 400 MHz): δ 2.80 (s, 3H), 2.50 (d, J=3.1 Hz, 1H), 2.26-2.16 (m, 2H), 2.07-2.00 (m, 1H), 1.86-1.30 (m, 15H), 1.24-1.16 (m, 1H), 1.01 (t, J=7.0 Hz, 3H); LCMS (m/z): 222 (M+1).

### Example 12

### N-d₂-methyl-7a-d₃-methyloctahydro-4,7-methano-1H-inden-3a-amine hydrochloride

A solution of spiro[bicyclo[2.2.1]heptane-2,1′-cyclobutan]-3-one (5.20 g, 34.7 mmol) in THF (250 mL), in a 500 mL flask, was stirred at 25° C. while d₃-methylmagnesium iodide (60 mL of 1.0 M in diethyl ether, 60.0 mmol) was added via syringe over a period of 5 min. The reaction was aged overnight at ambient temperature. Since LCMS analysis indicated that a small amount of starting material remained, an additional 5 mL (5.0 mmol) of the Grignard reagent was added and the solution stirred an additional 24 h. The THF was then removed under reduced pressure, and saturated aqueous ammonium chloride (200 mL) and dichloromethane (200 mL) were added to the residue. After thorough mixing and subsequent separation of the phases, the water layer was extracted with dichloromethane (2×150 mL). The combined organic layers were concentrated under reduced pressure, to give 3-d₃-methylspiro[bicyclo[2.2.1]heptane-2,1′-cyclobutan]-3-ol, as a light yellow oil (6.0 g). This crude oil was carried on to the next step without further purification. ¹H NMR (CDCl₃, 400 MHz): δ 2.20-2.08 (m, 2H), 2.02-1.90 (m, 2H), 1.80-1.56 (m, 5H), 1.52-1.32 (m, 3H), 1.32-1.20 (m, 2H); GCMS (m/z): 170 (M+1).

The 3-d₃-methylspiro[bicyclo[2.2.1]heptane-2,1′-cyclobutan]-3-ol was placed in a 500 mL flask and combined with acetic acid (15 mL) and sodium cyanide (3.10 g, 63.3 mmol). The flask was sealed with a rubber septum. The mixture was stirred at ambient temperature for 15 min and then was cooled to 0° C. in an ice bath. Sulfuric acid (12 mL, 225 mmol) was added via a syringe over a 20 min period. The mixture was warmed slowly to ambient temperature and stirred overnight. The mixture was then diluted with water (250 mL) and extracted with dichloromethane (250 mL). The dichloromethane layer was washed with 2.0 M aqueous sodium hydroxide (200 mL) and then water (200 mL). Concentration of the organic layer gave a tan solid (7.10 g). The solid was purified on a silica gel column, eluting with mixtures of ethyl acetate in hexanes (25%-100% ethyl acetate). Concentration of selected fractions gave N-(7a-d₃-methyloctahydro-4,7-methano-1H-inden-3a-yl)formamide (5.50 g, 84.2% yield). GCMS (m/z): 197 (M+1).

The N-(7a-d₃-methyloctahydro-4,7-methano-1H-inden-3a-yl)formamide (5.50 g, 28.1 mmol) was dissolved in THF (50 mL) and was added to a 500 mL flask containing a stirred mixture of lithium aluminum deuteride (4.00 g, 95.2 mmol) in THF (200 mL) at 0° C. The addition took 15 min. The mixture was refluxed for 9 h and then cooled to ambient temperature, where it was stirred overnight. The reaction was quenched with 2.0 M aqueous sodium hydroxide (15 mL), and the resulting mixture was filtered through diatomaceous earth. Separation and concentration of the THF layer gave N-d2-methyl-7a-d3-methyloctahydro-4,7-methano-1H-inden-3a-amine, as a colorless oil (4.70 g, 91.0% yield). GCMS (m/z): 185 (M+1).

To a solution of the N-d2-methyl-7a-d3-methyloctahydro-4,7-methano-1H-inden-3a-amine (4.70 g, 25.5 mmol) in methanol (100 mL) was added concentrated hydrochloric acid (5.0 mL, 60 mmol). After stirring for 10 min at ambient temperature, the solution was concentrated under reduced pressure. The residue was dissolved in methanol (200 mL) and concentrated three successive times, leaving a tan solid. The solid was dried in a vacuum oven at 60° C. for 6 h, providing N-d2-methyl-7a-d3-methyloctahydro-4,7-methano-1H-inden-3a-amine hydrochloride (4.80 g, 85.5% yield). ¹H NMR (D₂O) δ 2.54 (s, 1H), 2.25 (s, 1H), 2.00-1.85 (m, 2H), 1.70-1.30 (m, 9H), 1.20-1.00 (m, 2H); GCMS (m/z): 185 (M+1).

### Example 13

### 7a-(hydroxymethyl)octahydro-4,7-methano-1H-inden-3a-amine hydrochloride

To a solution of 1-cyclopentene-1,2-dicarboxylic anhydride (3.0 g, 22 mmol) in dry THF (10 mL) cooled to 0° C., was added freshly distilled cyclopentadiene (10 mL) and aluminum trichloride (80 mg, 0.60 mmol). The reaction was stirred for 30 min at 0° C. and then placed in a freezer at 0° C.-5° C. for 14 h. The reaction was then diluted with diethyl ether (50 mL) and washed with saturated aqueous sodium chloride (10 mL). The organic layer was separated, dried over anhydrous sodium sulfate, and filtered. The filtrate was concentrated under reduced pressure to give a solid. The solid was washed with hexanes and filtered, to give hexahydro-1H-4,7-methano-3a,7a-(methanooxymethano)indene-8,10-dione (3.8 g, 86% yield).

To a solution of hexahydro-1H-4,7-methano-3a,7a-(methanooxymethano)indene-8,10-dione (2.5 g, 12 mmol) in methanol (20 mL) was added 0.2 g 10% Pd/C (wet). This mixture was shaken under a hydrogen atmosphere (50 psi) for 16 h at ambient temperature. The mixture was then filtered through a pad of diatomaceous earth, and the filter cake was washed with methanol. The filtrate was then concentrated, and the residue vacuum dried to yield a lightly colored solid. The solid was dissolved in dry methanol (50 mL) and cooled in an ice bath. Sodium methoxide in methanol (13 mL of 25%, 48 mmol) was added to the reaction. The reaction was warmed to ambient temperature and stirred for 16 h. The mixture was concentrated by rotary evaporation, and the residue was partitioned between 6.0 M hydrochloric acid (20 mL) and dichloromethane (50 mL). The dichloromethane layer was separated, and the aqueous layer washed with dichloromethane (2×20 mL). The combined dichloromethane layers were passed through a phase separator and concentrated, to yield 7a-(methoxycarbonyl)octahydro-4,7-methano-1H-indene-3a-carboxylic acid as a light brown solid (2.9 g, ˜100% yield).

To a stirred solution of 7a-(methoxycarbonyl)octahydro-4,7-methano-1H-indene-3a-carboxylic acid (2.9 g, 12 mmol) and triethylamine (1.5 g, 15 mmol) in dry toluene (40 mL) cooled in an ice bath, was added diphenyl phosphoryl azide (3.5 g, 13 mmol). The reaction was warmed to 90° C. and stirred for 4 h.

The reaction was then cooled and concentrated by rotary evaporation. The residue was purified by silica gel column chromatography, eluting with a 0-20% ethyl acetate in hexanes gradient over 12 column volumes. Selected fractions were combined and concentrated to dryness, yielding methyl 7a-isocyanatooctahydro-4,7-methano-1H-indene-3a-carboxylate as a white solid (1.5 g, 52% yield).

A solution of methyl 7a-isocyanatooctahydro-4,7-methano-1H-indene-3a-carboxylate (1.0 g, 4.3 mmol) in THF (10 mL) was added to an ice-bath cooled mixture of lithium aluminum hydride (8.5 mL of 2 M in THF, 17 mmol) and THF (10 mL). After addition, the reaction was warmed to 50° C. and kept there for 16 h. The reaction was then cooled in an ice-bath and quenched with careful addition of water until a white slurry formed. The slurry was stirred in an ice-bath for 4 h before being filtered through a bed of diatomaceous earth. The filter cake was washed with ethyl acetate. The combined filtrates were washed with 6 M aqueous hydrochloric acid (3×15 mL), and the aqueous washes were combined and concentrated on a rotary evaporator to dryness. The resulting solid was dissolved with heating in 2-propanol, and diethyl ether was added until a precipitate was observed. The slurry was cooled in an ice-bath for 2 h, and the solids were collected by filtration and washed with ether. A second crop of material was isolated from the mother liquors after reducing volume and standing at ambient temperature for 24 h. The isolated solids were dried to yield 7a-(hydroxymethyl)octahydro-4,7-methano-1H-inden-3a-amine hydrochloride (0.64 g, 64% yield). ¹H NMR (400 MHz, D₂O): δ 3.60 (d, J=1 Hz, 1H), 3.48 (d, J=1 Hz, 1H), 2.55 (s, 3H), 2.31 (d, J=3 Hz, 1H), 1.97 (bs, 2H), 1.87 (d, J=6 Hz, 1H), 1.65-1.34 (m, 8H), 1.19-1.07 (m, 2H); LCMS (m/z): 196 (M+1).

### Example 14

### (3aS,4S,7R,7aS)-N,N,7a-trimethyloctahydro-4,7-methano-1H-inden-3a-amine hydrochloride

To a mixture of (3aS,4S,7R,7aS)-N,7a-dimethyloctahydro-4,7-methano-1H-inden-3a-amine hydrochloride (0.13 g, 0.60 mmol) and potassium carbonate (0.42 g, 3.0 mmol) in acetonitrile (5 mL) was added iodomethane (0.86 g, 6.0 mmol). The reaction was capped tightly and stirred at 40° C. for 3 h. The reaction was filtered, and the filtrate was concentrated. The residue was partitioned between dichloromethane (30 mL) and water (50 mL). The organic layer was separated, dried over sodium sulfate and filtered. To filtered organic layer was added concentrated hydrochloric acid (0.3 mL), and the mixture was concentrated to dryness, leaving (3aS,4S,7R,7aS)-N,N,7a-trimethyloctahydro-4,7-methano-1H-inden-3a-amine hydrochloride (104 mg), as a white solid. ¹H NMR (CD₃OD, 400 MHz): δ 2.88 (s, 3H), 2.86 (s, 3H), 2.45-2.38 (m, 2H), 1.97 (brs, 1H), 1.87-1.46 (m, 10H), 1.34-1.26 (m, 4H); LCMS (m/z): 194 (M+1).

### Example 15

### (3aR,4R,7S,7aR)-N,N,7a-trimethyloctahydro-4,7-methano-1H-inden-3a-amine hydrochloride

To a mixture of (3aR,4R,7S,7aR)-N,7a-dimethyloctahydro-4,7-methano-1H-inden-3a-amine hydrochloride (0.18 g, 0.83 mmol) and potassium carbonate (0.58 g, 4.2 mmol) in acetonitrile (5 mL) was added iodomethane (1.2 g, 8.4 mmol). The reaction was capped tightly and stirred at 40° C. for 3 h. The reaction was filtered, and the filtrate was concentrated. The residue was partitioned between dichloromethane (40 mL) and water (50 mL). The organic layer was separated, dried over sodium sulfate and filtered. To filtered organic layer was added concentrated hydrochloric acid (0.5 mL), and the mixture was concentrated to dryness, leaving (3aR,4R,7S,7aR)-N,N,7a-trimethyloctahydro-4,7-methano-1H-inden-3a-amine hydrochloride (181 mg), as a white solid. ¹H NMR (CD₃OD, 400 MHz): δ 2.88 (s, 3H), 2.86 (s, 3H), 2.45-2.38 (m, 2H), 1.97 (brs, 1H), 1.87-1.46 (m, 10H), 1.34-1.26 (m, 4H); LCMS (m/z): 194 (M+1).

### VI. Biological Assays

### Characterization of Interactions at Nicotinic Acetylcholine Receptors Materials and Methods

Cell Lines.

SH-EP1-human α4β2 (Eaton et al., 2003), cell lines were obtained from Dr. Ron Lukas (Barrow Neurological Institute). Cells were maintained in proliferative growth phase in Dulbecco's modified Eagle's medium (Invitrogen, Carlsbad, Calif.) with 10% horse serum (Invitrogen), 5% fetal bovine serum (HyClone, Logan Utah), 1 mM sodium pyruvate, 4 mM L-glutamine. For maintenance of stable transfectants, the α4β2 cell media was supplemented with 0.25 mg/mL zeocin and 0.13 mg/mL hygromycin B.

CHO-human α7 cells (obtained from ChanTest, Cleveland, Ohio, catalog #CT6201) were maintained in proliferative growth phase in Ham's F12 (VWR) with 10% fetal bovine serum (Invitrogen), 0.25 mg/mL geneticin; 0.4 mg/ml zeocin. The amino acid sequences encoded by the transfected cDNA constructs used to generate the CHO-human α7 cells are identical to the translated sequences for GenBank accession numbers NM_{—}000746.4 (α7) and NM_{—}024557.4 (hRIC3).

CHO-human α3β4 cells (obtained from ChanTest, Cleveland, Ohio, catalog #CT6021) were maintained in proliferative growth phase in Ham's F12 (VWR) with 10% fetal bovine serum (Invitrogen), 0.25 mg/mL geneticin; 0.4 mg/ml zeocin. The amino acid sequences encoded by the transfected cDNA constructs used to generate the CHO-human α3β4 cells are identical to the translated sequences for GenBank accession numbers NM_{—}000743.2 and NM_{—}000750.3, respectively.

### Receptor Binding Assays

Preparation of Membranes from Clonal Cell Lines.

Cells were harvested in ice-cold PBS, pH 7.4, then homogenized with a Polytron (Kinematica GmbH, Switzerland). Homogenates were centrifuged at 40,000 g for 20 minutes (4° C.). The pellet was re-suspended in PBS and protein concentration determined using the Pierce BCA Protein Assay kit (Pierce Biotechnology, Rockford, Ill.).

Competition Binding to Receptors in Membrane Preparations.

Binding to nicotinic receptors was assayed on membranes using standard methods adapted from published procedures (Lippiello and Fernandes 1986; Davies et al., 1999). In brief, membranes were reconstituted from frozen stocks and incubated for 2 h on ice in 150 μl assay buffer (50 mM Tris, 154 mM NaCl, pH 7.4) in the presence of competitor compound (0.001 nM to 100 μM) and radioligand. [³H]-nicotine (L-(−)-[N-methyl-³H]-nicotine, 69.5 Ci/mmol, Perkin-Elmer Life Sciences, Waltham, Mass.) was used for human α4β2 binding studies. [³H]-epibatidine (52 Ci/mmol, Perkin-Elmer Life Sciences) was used for binding studies at the other nicotinic receptor subtypes. Membrane source, radioligand, and radioligand concentration for each receptor target are listed in Table 3. Incubation was terminated by rapid filtration on a multimanifold tissue harvester (Brandel, Gaithersburg, Md.) using GF/B filters presoaked in 0.33% polyethyleneimine (w/v) to reduce non-specific binding. Filters were washed 3 times with ice-cold assay buffer and the retained radioactivity was determined by liquid scintillation counting.

TABLE 1
Binding Parameters

| | | | Radioligand |
|---|---|---|---|
| | | | concentration |
| Binding target | Membrane Source | Radioligand | (nM) |
| Nicotinic, human | SH-EP1-Human | [³H]nicotine | 2 |
| α4β2 | α4β2 cells | | |
| Nicotinic, human | CHO Human | [³H]TC-12018 | 0.5 |
| α7 | α7 | | |
| Nicotinic, human | SH-SY5Y cells | [³H]epibatidine | 1 |
| α3β4α5 | | | |
| Nicotinic, human | CHO Human | [³H]epibatidine | 1 |
| α3β4 | α3β4 | | |

Binding Data Analysis.

Binding data were expressed as percent total control binding. Replicates for each point were averaged and plotted against the log of drug concentration. The IC₅₀ (concentration of the compound that produces 50% inhibition of binding) was determined by least squares non-linear regression using GraphPad Prism software (GraphPAD, San Diego, Calif.). Ki was calculated using the Cheng-Prusoff equation (Cheng and Prusoff, 1973).

### Calcium Flux Functional Assays

Forty-eight hours prior to each experiment, cells were plated in 96 well black-walled, clear bottom plates (Corning, Corning, N.Y.) at 60-100,000 cells/well. On the day of the experiment, growth medium was gently removed, 200 μL 1×FLIPR Calcium 4 Assay reagent (Molecular Devices, Sunnyvale, Calif.) in assay buffer (20 mM HEPES, 7 mM TRIS base, 4 mM CaCl₂, 5 mM D-glucose, 0.8 mM MgSO₄, 5 mM KCl, 0.8 mM MgCl₂, 120 mM N-methyl D-glucamine, 20 mM NaCl, pH 7.4 for SH-EP1-human α4β2 cells or 10 mM HEPES, 2.5 mM CaCl₂, 5.6 mM D-glucose, 0.8 mM MgSO₄, 5.3 mM KCl, 138 mM NaCl, pH 7.4 with TRIS-base for all other cell lines) was added to each well and plates were incubated at 37° C. for 1 hour (29° C. for the 29° C.-treated SH-EP1-human α4β2 cells). For inhibition studies, competitor compound (10 pM-10 μM) was added at the time of dye addition. The plates were removed from the incubator and allowed to equilibrate to room temperature. Plates were transferred to a FLIPR Tetra fluorometric imaging plate reader (Molecular Devices) for addition of compound and monitoring of fluorescence (excitation 485 nm, emission 525 nm). The amount of calcium flux was compared to both a positive (nicotine) and negative control (buffer alone). The positive control was defined as 100% response and the results of the test compounds were expressed as a percentage of the positive control. For inhibition studies, the agonist nicotine was used at concentrations of 1 μM for SH-EP1-human α4β2 treated at 29° C. (HS), 10 μM for SH-EP1-human α4β2 maintained at 37° C. (LS), and 20 μM for SH-SY5Y cells or CHO_human α3β4.

### Patch Clamp Electrophysiology

Cell Handling.

After removal of GH4C1-rat T6'S α7 cells from the incubator, medium was aspirated, cells trypsinized for 3 minutes, gently triturated to detach them from the plate, washed twice with recording medium, and re-suspended in 2 ml of external solution (see below for composition). Cells were placed in the Dynaflow chip mount on the stage of an inverted Zeiss microscope (Carl Zeiss Inc., Thornwood, N.Y.). On average, 5 minutes was necessary before the whole-cell recording configuration was established. To avoid modification of the cell conditions, a single cell was recorded per single load. To evoke short responses, compounds were applied for 0.5 s using a Dynaflow system (Cellectricon, Inc., Gaithersburg, Md.), where each channel delivered pressure-driven solutions at either 50 or 150 psi.

Electrophysiology.

Conventional whole-cell current recordings were used. Glass microelectrodes (5-10 MΩ resistance) were used to form tight seals (>1 GΩ) on the cell surface until suction was applied to convert to conventional whole-cell recording. The cells were then voltage-clamped at holding potentials of −60 mV, and ion currents in response to application of ligands were measured. Whole-cell currents recorded with an Axon 700A amplifier were filtered at 1 kHz and sampled at 5 kHz by an ADC board 1440 (Molecular Devices). Whole-cell access resistance was less than 20 MΩ. Data acquisition of whole-cell currents was done using a Clampex 10 (Molecular Devices, Sunnyvale, Calif.), and the results were plotted using Prism 5.0 (GraphPad Software Inc., San Diego, Calif.). The experimental data are presented as the mean±S.E.M., and comparisons of different conditions were analyzed for statistical significance using Student's t and Two Way ANOVA tests. All experiments were performed at room temperature (22±1° C.). Concentration-response profiles were fit to the Hill equation and analyzed using Prism 5.0.

Solutions and Drug Application.

The standard external solution contained: 120 mM NaCl, 3 mM KCl, 2 mM MgCl₂, 2 mM CaCl₂, 25 mM D-glucose, and 10 mM HEPES and was adjusted to pH 7.4 with Tris base. Internal solution for whole-cell recordings consisted of: 110 mM Tris phosphate dibasic, 28 mM Tris base, 11 mM EGTA, 2 mM MgCl₂, 0.1 mM CaCl₂, and 4 mM Mg-ATP, pH 7.3. (Liu et al., 2008). To initiate whole-cell current responses, compounds were delivered by moving cells from the control solution to agonist-containing solution and back so that solution exchange occurred within ˜50 ms (based on 10-90% peak current rise times). Intervals between compound applications (0.5-1 min) were adjusted specifically to ensure the stability of receptor responsiveness (without functional rundown), and the selection of pipette solutions used in most of the studies described here was made with the same objective. (−)-Nicotine and acetylcholine (ACh), were purchased from Sigma-Aldrich (St. Louis, Mo.). All drugs were prepared daily from stock solutions.

To determine the inhibition of ACh induced currents by compounds of the present invention, we established a stable baseline recording applying 70 μM ACh (usually stable 5-10 consecutive applications). Then ACh (70 μM) was co-applied with test compound in a concentration range of 1 nM to 10 μM. Since tail of the current (current measured at the end of 0.5 s ACh application) underwent the most profound changes, inhibition and recovery plots represent amplitude of tail current.

### Cross-Comparisons, Electrophysiology

Subclonal Human Epithelial-hα4β2 Cells. Established techniques were used to introduce human α4 (S452) and β2 subunits (kindly provided by Dr. Ortrud Steinlein, Institute of Human Genetics, University Hospital, Ludwig-Maximilians-Universitat, Munich, Germany) and subcloned into pcDNA3.1-zeocin and pcDNA3.1-hygromycin vectors, respectively, into native NNR-null SHEP1 cells to create the stably transfected, monoclonal subclonal human epithelial (SH-EP1)-hα4β2 cell line heterologously expressing human α4β2 receptors. Cell cultures were maintained at low passage numbers (1-26 from frozen stocks to ensure the stable expression of the phenotype) in complete medium augmented with 0.5 mg/ml zeocin and 0.4 mg/ml hygromycin (to provide a positive selection of transfectants) and passaged once weekly by splitting the just-confluent cultures 1:20 to maintain cells in proliferative growth. Reverse transcriptase-polymerase chain reaction, immunofluorescence, radioligand-binding assays, and isotopic ion flux assays were conducted recurrently to confirm the stable expression of α4β2 NNRs as message, protein, ligand-binding sites, and functional receptors.

Cell Handling.

Similar to that presented hereinabove, after removal from the incubator, the medium was aspirated, and cells were trypsinized for 3 min, washed thoroughly twice with recording medium, and resuspended in 2 ml of external solution (see below for composition). Cells were gently triturated to detach them from the plate and transferred into 4-ml test tubes from which cells were placed in the Dynaflow chip mount on the stage of an inverted Zeiss microscope (Carl Zeiss Inc., Thornwood, N.Y.). On average, 5 min was necessary before the whole-cell recording configuration was established. To avoid modification of the cell conditions, a single cell was recorded per single load. To evoke short responses, agonists were applied using a Dynaflow system (Cellectricon, Inc., Gaithersburg, Md.), where each channel delivered pressure-driven solutions at either 50 or 150 psi.

Electrophysiology.

Similar to that present hereinabove, conventional whole-cell current recordings, together with a computer-controlled Dynaflow system (Cellectricon, Inc.) for fast application and removal of agonists, were used in these studies. In brief, the cells were placed in a silicon chip bath mount on an inverted microscope (Carl Zeiss Inc.). Cells chosen for analysis were continuously perfused with standard external solution (60 μl/min). Glass microelectrodes (3-5 MΩ resistance between the pipette and extracellular solutions) were used to form tight seals (1 GΩ) on the cell surface until suction was applied to convert to conventional whole-cell recording. The cells were then voltage-clamped at holding potentials of −60 mV, and ion currents in response to application of ligands were measured. Whole-cell currents recorded with an Axon 700A amplifier were filtered at 1 kHz and sampled at 5 kHz by an ADC board 1440 (Molecular Devices) and stored on the hard disk of a PC computer. Whole-cell access resistance was less than 20 MΩ. Data acquisition of whole-cell currents was done using a Clampex 10 (Molecular Devices, Sunnyvale, Calif.), and the results were plotted using Prism 5.0 (GraphPad Software Inc., San Diego, Calif.). The experimental data are presented as the mean±S.E.M., and comparisons of different conditions were analyzed for statistical significance using Student's t tests. All experiments were performed at room temperature (22±1° C.). Concentration-response profiles were fit to the Hill equation and analyzed using Prism 5.0. No differences in the fraction of responsive cells could be detected among experimental conditions. More than 90% of the cells responded to acetylcholine (ACh), and every cell presenting a measurable current was taken into account. Cells were held at −60 mV throughout the experiment. All drugs were prepared daily from stock solutions.

Neuronal α4β2 receptor dose-response curves could be described by the sum of two empirical Hill equations comparable with methods described previously (Covernton and Connolly, 2000):

y=Iₘₐₓ·(α1/(1+(EC₅₀H/x)^{xn})+(1−α1)/(1+(EC₅₀L/x)^{xn}) (1)

where Imax is the maximal current amplitude, and x is the agonist concentration. EC50H, nH1, and al are the half-effective concentration, the Hill coefficient, and the percentage of receptors in the HS state. EC50L and nH2 are the half-effective concentration and the Hill coefficient in the LS state. In some cases, a single Hill equation,

y=Iₘₐₓ×[1/(1+(EC₅₀/x)^{nH})]

was used for comparison of the fit with eq. 1. Imax, EC50, and nH have the same meanings.

The time course of open-channel block of responses to α4β2 agonists was analyzed using a monoexponential equation of the form:

Y=A·exp(−t/τ)−B (2)

where y is the current (in picoamperes), A is the control maximum peak current (in picoamperes), τ is the time constant (in milliseconds), B is the current at equilibrium (in picoamperes), and t is the time (in milliseconds).

Solutions and Drug Application.

The standard external solution contained: 120 mM NaCl, 3 mM KCl, 2 mM MgCl₂, 2 mM CaCl₂, 25 mM D-glucose, and 10 mM HEPES and was adjusted to pH 7.4 with Tris base. In the experiments, ACh was applied as an agonist without atropine because our experimental data showed that 1 μM atropine sulfate did not affect ACh-induced currents (not shown) and because atropine itself has been reported to lock nicotinic receptors (Liu et al., 2008). For all conventional whole-cell recordings, Tris electrodes were used and filled with solution containing: 110 mM Tris phosphate dibasic, 28 mM Tris base, 11 mM EGTA, 2 mM MgCl₂, 0.1 mM CaCl₂, and 4 mM Mg-ATP, pH 7.3. To initiate whole-cell current responses, nicotinic agonists were delivered by moving cells from the control solution to agonist-containing solution and back so that solution exchange occurred within ˜50 ms (based on 10-90% peak current rise times). Intervals between drug applications (0.5-1 min) were adjusted specifically to ensure the stability of receptor responsiveness (without functional rundown), and the selection of pipette solutions used in most of the studies described here was made with the same objective. The drugs used in the present studies, including (−)-nicotine and ACh, were purchased from Sigma-Aldrich (St. Louis, Mo.).

### Tabulated Summary

As shown in Table 2, compounds representative of the present invention typically exhibit inhibition constants (Ki values) for human α4β2, α7, and ganglionic receptor subtypes in the 1-100 mM range, indicating a low affinity for the orthosteric binding sites (i.e. the binding site of the competitive agonist) of these receptor subtypes. The data in Table 4, however, also illustrates that compounds representative of the present invention effectively inhibit ion flux for these receptor subtypes, with typical IC₅₀ values of less than about 2 mM and typical Iₘₐₓ values of >95%. Taken together, this data demonstrates that the compounds representative of this invention are effective at inhibiting ion flux mediated by these receptor subtypes through a mechanism that does not involve binding at the orthosteric sites.

TABLE 2

| | | | | | | | | | Human | Human | | |
|---|---|---|---|---|---|---|---|---|---|---|---|---|
| | Hu- | Hu- | Human | Human | Human | Human | Human | Human | CHO | CHO | Human | Human |
| | man | man | CHO | Gan- | α4β2 | α4β2 | α4β2 | α4β2 | α3β4 | α3β4 | Ganglion | Ganglion |
| | α4β2 | CHO | α3β4 | glion | Ca Flux | Ca Flux | Ca Flux | Ca Flux | Ca Flux | Ca Flux | Ca Flux | Ca Flux |
| | Ki | α7 Ki | Ki | Ki | IC50 [29C/ | Imax [29C/ | IC50 [37C/ | Imax [37C/ | IC50 | Imax | IC50 | Imax |
| Structure | (nM) | (nM) | (nM) | (nM) | HS] (nM) | HS] (% inh) | LS] (nM) | HS] (% inh) | (nM) | (% inh) | (nM) | (% inh) |
| 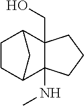 | 31000 | | | 89000 | 370 | 99 | 380 | 90 | | | 200 | 91 |
| 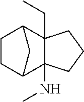 | 34000 | | 75000 | 100000 | 430 | 100 | 170 | 97 | 220 | 97 | 20 | 98 |
| 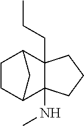 | 29000 | | 73000 | 52000 | 210 | 100 | 170 | 96 | 130 | 96 | 30 | 98 |
| 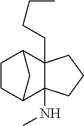 [SMILES: CCCCC12CCCC1(NC)C1CCC2C1] | 73000 | | 74000 | 100000 | 460 | 99 | 140 | 96 | 300 | 96 | 24 | 93 |
| 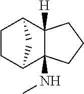 | 33000 | | | 100000 | 550 | 96 | 340 | 96 | 490 | 97 | 380 | 95 |
| 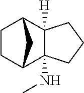 | 51000 | | 80000 | 70000 | 380 | 97 | 380 | 96 | 660 | 97 | 490 | 95 |
| 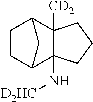 | 26000 | 100000 | 100000 | | | | 76 | 95 | | | 83 | 99 |
| 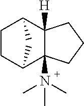 | 8600 | | 13000 | | | | 380 | 100 | | | 1800 | 97 |
| 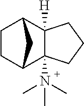 | 6900 | | | | | | 260 | 98 | 940 | 94 | | |
| 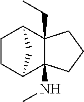 | 26000 | 79000 | 56000 | | 600 | 98 | 5000 | 93 | 170 | 97 | | |
| 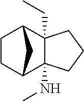 | 32000 | 100000 | 53000 | | 510 | 100 | 6900 | 94 | 120 | 97 | | |
| 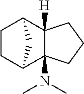 | 47000 | | 16000 | | 410 | 99 | 410 | 98 | 230 | 97 | | |
| 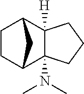 | 20000 | | 13000 | | 510 | 96 | 500 | 98 | 190 | 96 | | |
| 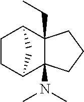 | 33000 | | 91000 | | | | 710 | 98 | 240 | 95 | | |
| 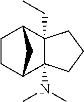 | 64000 | | 70000 | | | | 370 | 99 | 200 | 96 | | |

The specific pharmacological responses observed may vary according to and depending on the particular active compound selected or whether there are present pharmaceutical carriers, as well as the type of formulation and mode of administration employed, and such expected variations or differences in the results are contemplated in accordance with practice of the present invention.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

1. A compound of Formula I: 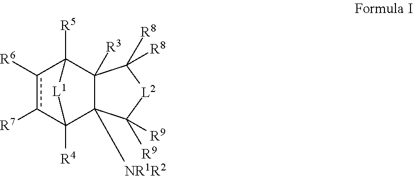
wherein
each of R¹ and R² individually is H, C₁₋₆ alkyl, or aryl-substituted C₁₋₆ alkyl, or R¹ and R² combine with the nitrogen atom to which they are attached to form a 3- to 8-membered ring, which ring may be optionally substituted with C₁₋₆ alkyl, aryl, C₁₋₆ alkoxy, or aryloxy substituents;
R³ is H, C₁₋₆ alkyl, hydroxyl substituted C₁₋₆ alkyl, or C₁₋₆ alkoxy-substituted C₁₋₆ alkyl;
each of R⁴, R⁵, R⁶, and R⁷ individually is H, C₁₋₆ alkyl, or C₁₋₆ alkoxy;
each R⁶ individually is H, C₁₋₆ alkyl, or C₁₋₆ alkoxy;
each R⁹ individually is H, C₁₋₆ alkyl, or C₁₋₆ alkoxy;
each L¹ and L² individually is a linker species selected from the group consisting of CR¹⁰R¹¹, CR¹⁰R¹¹CR¹²R¹³, and O;
each of R¹⁹, R¹¹, R¹², and R¹³ individually is hydrogen or C₁₋₆ alkyl;
or a pharmaceutically acceptable salt thereof.

2. The compound of claim 1, wherein R¹ is H and R² is C₁₋₆ alkyl.

3. The compound of claim 1, wherein R³ is C₁₋₆ alkyl or hydroxyl-substituted C₁₋₆ alkyl.

4. The compound of claim 1, wherein each of R⁴, R⁵, R⁶, R⁷, R⁸, and R⁹ is H.

5. The compound of claim 1, wherein each of L¹ and L² is CR¹⁰R¹¹, and each of R¹⁰ and R¹¹ is hydrogen.

6. A pharmaceutical composition comprising a compound as claimed in claim 1 and a pharmaceutically acceptable carrier.

7. A method for the treatment or prevention of a disease or condition mediated by a neuronal nicotinic receptor comprising the administration of a compound as claimed in claim 1.

8. The method of claim 7, wherein the disease or condition is IBS-D, OAB, nicotine addiction, smoking cessation, depression, major depressive disorder, or hypertension.

9. (canceled)

10. (canceled)

11. (canceled)

12. (canceled)

13. (canceled)
